(19) United States
(12) Patent Application Publication
(10) Pub. No.: US 2017/0247674 A1
(43) Pub. Date: Aug. 31, 2017

(54) CONJUGATES FOR DELIVERY TO MITOCHONDRIA

(71) Applicant: The Forsyth Institute, Cambridge, MA (US)

(72) Inventors: Heike Boisvert, Somerville, MA (US); Margaret Duncan, Brookline, MA (US)

(21) Appl. No.: 15/519,369

(22) PCT Filed: Oct. 14, 2015
(86) PCT No.: PCT/US2015/055583

§ 371 Date: Apr. 14, 2017

Related U.S. Application Data
(60) Provisional application No. 62/063,938, filed on Oct. 14, 2014.
(60) Provisional application No. 62/063,928, filed on Oct. 14, 2014.

(51) Int. Cl. C12N 9/52; A61K 38/48

(52) U.S. Cl. CPC C12N 9/52; A61K 47/48107; A61K 38/4873; C07K 2319/07; C07K 2319/10; C07K 2319/31; C07K 2319/30

(57) ABSTRACT

The present disclosure relates generally to conjugates and their uses and production. More particularly, it concerns conjugates comprising an A44 or K39 gingipain adhesion peptide, or fragment thereof (e.g., an A44/K39 peptide portion) covalently attached to a heterologous agent that is capable of entering a cell by endocytosis and translocating to mitochondria.

### RELATED APPLICATIONS

This application claims the benefit of priority from U.S. provisional application No. 62/063,938, filed Oct. 14, 2014, and U.S. provisional application No. 62/063,928, filed Oct. 14, 2014. The disclosures of each of the foregoing applications are hereby incorporated by reference in their entirety.

### GOVERNMENT SUPPORT

This disclosure was made with government support under Grant Numbers RO1 DE010510 and RO1 DE015931 awarded by the National Institutes of Health. The government has certain rights in the disclosure.

### FIELD OF THE DISCLOSURE

The present disclosure relates generally to conjugates and their uses and production. More particularly, it concerns conjugates comprising an A44 or K39 gingipain adhesin peptide, or fragment thereof (e.g., an A44/K39 peptide portion) associated with a heterologous agent that is capable of entering a cell, such as by endocytosis, and translocating to mitochondria. The present disclosure also relates generally to methods of using these conjugates for targeted drug delivery, treatment, diagnostic, and research methods in cells and/or in subjects, particularly in cells and subjects characterized by mitochondrial dysfunction.

### BACKGROUND

Mitochondrial diseases are a clinically heterogeneous group of disorders associated with deficiency and/or dysfunction of mitochondria. Because mitochondria play a central role in cellular functions which include the generation and regulation of energy metabolism and the regulation of cell death and survival pathways, mitochondrial diseases can affect essentially all organ systems. Many mitochondrial diseases have a genetic component and can arise through defects either in nuclear or mitochondrial DNA. Over 200 defects in mitochondrial DNA have been identified as pathogenic.

One therapeutic category of mitochondrial diseases is associated with cellular degeneration, which is often mediated by oxidative stress and apoptotic mechanisms. Cardiovascular diseases like atherosclerosis, ischemia/reperfusion injury, heart failure, stroke, and traumatic brain injury; neurodegenerative diseases like Alzheimer's disease, Parkinson's disease, Huntington's disease, amyotrophic lateral sclerosis (ALS), autism, and muscular dystrophy; chronic autoimmune inflammatory diseases like rheumatoid arthritis; metabolic diseases like diabetes and obesity; and aging could all be considered in this category. Another therapeutic category of mitochondrial diseases is associated with hyperproliferative states, e.g., cancers.

The Center for Disease Control and the Foundation for Mitochondrial Medicine estimate that the incidence of mitochondrial disease is 1-5 in 10,000. However, this number may underestimate the true number of people with mitochondrial disease.

One barrier for developing effective therapeutic, diagnostic and research tools for addressing mitochondrial diseases is the lack of efficient strategies and reagents for targeting mitochondria. Given the prevalence of mitochondrial disease, there is a need for mitochondria-targeting strategies since the efficacy of an agent is likely to be highly dependent on the ability, not only to deliver agents into cells, but also to effectively deliver agents to and into the mitochondria. The delivery problem is particularly acute for mitochondria-targeted agents because the mitochondrial membranes are generally highly impermeable. Accordingly, effective transit across one or both mitochondrial membranes can be difficult to achieve.

### SUMMARY OF THE DISCLOSURE

The present disclosure provides compositions and methods of use and production. The present disclosure provides conjugates to address the need for systems suitable to deliver agents, such as therapeutic, diagnostic and research agents, into cells and to promote trafficking or translocation to mitochondria. Such conjugates (e.g., conjugates of the disclosure) are useful in a variety of in vitro and in vivo methods, and such methods are contemplated and described herein. Any of the conjugates described herein can be described using any combination of structural and/or functional features of the A44/K39 peptide portion and/or the heterologous agent portion. Moreover, conjugates include any combination of A44/K39 peptide portions and heterologous agent portions, including conjugates in which the portions are associated via a peptide bond or chemical conjugation, and whether in the presence or absence of a linker interconnecting the portions. Numerous examples are provided herein. Any such conjugates may be provided as isolated and/or purified compounds and/or as a composition comprising the conjugate formulated with one or more pharmaceutically acceptable carriers and/or excipients. Moreover, any of the conjugates (including compositions) may be used in any of the methods of the disclosure described herein. Accordingly, the disclosure contemplates combinations of one or more of any of the aspects and embodiments described herein.

The disclosure provides conjugates, as well as methods for making and using such conjugates comprising i) an A44 or K39 peptide, or functional fragment thereof capable of entering a cell by endocytosis and translocating to mitochondria (e.g., an A44/K39 peptide portion), and ii) a heterologous agent (e.g., the heterologous agent portion) covalently associated therewith, wherein the heterologous agent is not a fluorescent protein or a fluorescent dye; and wherein the conjugate does not comprise a catalytic domain of a gingipain protein. In other words, the disclosure provides a conjugate composed of at least two components: an A44 or K39 derived peptide component (one portion of a conjugate; referred to as a A44/K39 peptide portion) that promotes internalization into cells and trafficking to the mitochondria, and a heterologous agent component (a second portion of a conjugate; referred to as a heterologous agent portion) that “piggybacks” into the cell via its covalent attachment with the aforementioned A44/K39 component. By heterologous it is meant that the heterologous agent portion comprises an agent that is not naturally associated with the A44 or K39 gingipain peptide portion. Similarly, in certain embodiments, conjugates of these two portions are interconnected via an association other than a covalent association. Accordingly, any of the features of conjugates described herein are similarly applicable to such conjugates associated or complexed non-covalently and such conjugates are contemplated. Any conjugate having any A44/K39 peptide portion, as described herein, and any heterologous agent portion is referred to as a conjugate of the disclosure. Also provided are conjugates comprising (i) an A44/K39 peptide portion and (ii) a heterologous agent, wherein the A44/K39 peptide portion and/or conjugate has one or more functions of the native, full length A44 or K39 peptide. By way of example, the A44/K39 peptide portion may comprise or consist of a fragment of full length A44 or K39 that has one or more functions of the native, full length A44 or K39 peptide. In certain embodiments, the A44/K39 peptide portion and/or conjugate is capable of entering a cell by endocytosis and translocating to mitochondria. In certain embodiments, the A44/K39 peptide portion retains one or more functions of the native A44 or K39 peptide but lacks one or more other native functions. For example, in certain embodiments, the A44/K39 peptide portion is selected because it has the ability to enter cells by endocytosis and translocate to the nucleus, but it does not increase expression of one or more pro-survival factors or inhibit apoptosis.

In certain embodiments, the conjugate does not comprise a full length gingipain adhesin peptide other than an A44 or K39 peptide. In other words, although other gingipain adhesin peptides exist and are present in a native gingipain protein (see, for example, FIG. 1), and in fact, such other gingipain adhesin peptides are contiguous with the A44 or K39 peptide in a native gingipain, in certain embodiments, the conjugate does not include such another gingipain adhesin peptide (e.g., a full length A17 peptide). In certain embodiments, the conjugate does not comprise a functional fragment of another gingipain adhesin peptide. In certain embodiments, the conjugate comprises one or more other gingipain adhesin peptides or functional fragments thereof in addition to an A44 or K39 peptide.

In certain embodiments, the conjugate comprises a functional fragment (but not the full length peptide) of an A44 or K39 peptide capable of entering a cell by endocytosis and translocating to mitochondria (e.g., the A44/K39 peptide portion comprises or consists of a functional fragment of an A44 or K39 peptide). In certain embodiments, the conjugate comprises such a fragment and does not comprise the full length A44 and/or K39 peptide. In certain embodiments, the conjugate does not comprise the full length A44 and/or K39 peptide set forth in SEQ ID NO: 1 or SEQ ID NO: 2. In certain embodiments, the fragment of an A44 or K39 peptide capable of entering a cell by endocytosis and translocating to mitochondria comprises (or consists of) at least 243, 242, 241, 240, 239, 238, 237, 236, 235, 230, 225, 220, 215, 210, 205, 200, 195, 190, 185, 180, 175, 170, 165, 160, 155, 150, 145, 140, 135, 130, 125, 120, 115, 110, 105, 103, 100, 90, 80, 70, 60 or 50 amino acid residues, such as contiguous amino acid residues of a full length A44 or K39 peptide. In certain embodiments, the fragment comprises or consists of a peptide of any of the foregoing number of contiguous amino acid residues of full length A44 or K39. In certain embodiments, the fragment is a fragment missing a portion of the C or N-terminus of a full length A44 or K39 peptide. In certain embodiments, the fragment includes the N-terminus of a full length A44 or K39 peptide. In certain embodiments, the fragment of any of the foregoing minimal lengths includes the amino acid sequence: PNPNPNPN. In certain embodiments, the fragment of any of the foregoing minimal lengths and/or the conjugate includes the amino acid sequence: PNPNPNPNP or PNPNPNPNPNPN or PNPNPNPNPNPNP. In certain embodiments, the fragment of an A44 or K39 peptide capable of entering a cell by endocytosis and translocating to mitochondria comprises at least 243 amino acid residues. In certain embodiments, the fragment of an A44 or K39 peptide capable of entering a cell by endocytosis and translocating to mitochondria comprises at least 242 amino acid residues. In certain embodiments, the fragment of an A44 or K39 peptide capable of entering a cell by endocytosis and translocating to mitochondria comprises at least 150 amino acid residues. In certain embodiments, the fragment of an A44 or K39 peptide capable of entering a cell by endocytosis and translocating to mitochondria comprises at least 100, 90, 80, 70, 60 or 50 amino acid residues. These are exemplary of functional fragments. In certain embodiments, a suitable functional fragment retains one or more functions of a native, full length A44 or K39 peptide. Optionally, an A44/K39 peptide portion and/or conjugate may include an N-terminal methionine to, for example, facilitate expression.

The full length, native sequence of the A44 peptide is set forth in SEQ ID NO: 1. The full length, native sequence of the K39 peptide is set forth in SEQ ID NO.: 2. These are examples of A44/K39 peptide portions for use herein. We note that, for the purpose of facilitating expression, an A44/K39 peptide portion, including the A44 or K39 peptide may optionally include an N-terminal methionine. Peptides and fragments made and tested (see Examples) included such an N-terminal methionine. Similar examples are functional fragments of these native peptides and variants of these native peptides have at least 80% amino acid sequence identity, over the corresponding portion, to the native peptide. Suitable functional fragments and variants retain one or more functions of native full length A44 or K39, such as any one or more of: capable of entering epithelial cells and, optionally, other cell types; capable of entering epithelial cells and, optionally other cell types by endocytosis; capable of translocating to mitochondria; capable of transitting at least one mitochondrial membrane; capable of transitting both mitochondrial membranes; capable of promoting or increasing expression of one or more anti-apoptotic gene (e.g., bcl-2, bcl-XL, NFkB, and mcl-1); capable of decreasing or promoting decrease in expression of one or more pro-apoptotic genes (e.g., bax, bak and TNFa); capable of increasing or promoting increase in expression of one or more pro-survival genes (e.g., rhoA, stat3, and cIAP1). In certain embodiments, a suitable functional fragment or variant retains one or more of the foregoing functions but does not possess one or more other functions. For example, in certain embodiments, it may be desirable to use a fragment or variant of the A44 or K39 peptide that enters cells and translocates to mitochondria but does not increase expression of one or more anti-apoptotic genes. In certain embodiments, a suitable functional fragment or variant retains two or more of the foregoing functions, such as the ability to enter cells by endocytosis and the ability to translocate to mitochondria. In certain embodiments, the functional fragment or variant has at least one, two, three, four, or five, or even all of the foregoing characteristics. These characteristics may be evaluated in a suitable cell based assay.

Optionally, conjugates of the disclosure may include a methionine at the N-terminus of one or both of the A44/K39 peptide portions and heterologous agent portions to facilitate expression. Thus, for example, a conjugate of the disclosure may comprise the amino acid sequence set forth in SEQ ID NOs: 1 or 2 and further include a methionine N-terminal to this sequence.

In certain embodiments, the conjugate does not include a full length A44 or K39 peptide. In other words, the conjugate comprises a portion or fragment of an A44 or K39 peptide (e.g., the A44/K39 peptide portion is a functional fragment of the A44 or K39 peptide set forth in SEQ ID NO: 1 or 2). In certain embodiments, the functional fragment of an A44 or K39 peptide comprises at least 243, 242, 241, 240, 235, 230, 225, 220, 215, 210, 205, 200, 195, 190, 185, 180, 175, 170, 165, 160, 155, 150, 145, 140, 135, 130, 125, 120, 115, 110, 105, 100, 90, 80, 70, 60 or 50 amino acid residues, such as any of the foregoing number of residues of SEQ ID NO: 1 or 2. In certain embodiments, the fragment comprises 50-243 amino acid residues. In certain embodiments, the fragment of an A44 or K39 peptide comprises at least 240 or at least 242 or at least 243 amino acid residues. In certain embodiments, the fragment of an A44 or K39 peptide comprises at least 150 amino acid residues. In certain embodiments, the fragment of an A44 or K39 peptide comprises at least 100, 90, 80, 70, 60 or 50 amino acid residues. In certain embodiments, the fragment includes the N-terminus of an A44 or K39 peptide. Suitable fragments retain one or more functional characteristics of native, full length A44 or K39 peptide, for example, one or more of: capable of entering epithelial cells and, optionally, other cell types; capable of entering epithelial cells and, optionally other cell types by endocytosis; capable of translocating to mitochondria; capable of transitting at least one mitochondrial membrane: capable of transitting both mitochondrial membranes; capable of promoting or increasing expression of one or more anti-apoptotic gene (e.g., bcl-2, bcl-XL, NFkB, and mcl-1); capable of decreasing or promoting decrease in expression of one or more pro-apoptotic genes (e.g., bax, bak and TNFa); capable of increasing or promoting increase in expression of one or more pro-survival genes (e.g., rhoA, stat3, and cIAP1). Any of these functional fragments are examples of an A44/K39 peptide portion suitable for use in a conjugate of the disclosure. We note that “retaining” a particular functional attribute does not require that the functional fragment or variant has the identical activity level relative to the the native A44 or K39 peptide. Rather, retaining a particular functional attribute indicates that the fragment or variant retains sufficient activity (e.g., at least 50%, 60%, 70%, 75%, 80%). In certain embodiments, the functional fragment or variant has an activity (vis-à-vis a particular functional feature) that is substantially the same as the native peptide. Functional activity is measured in a suitable in vitro assay under standard conditions, such as the assays provided herein.

In certain embodiments, the fragment is a fragment missing a portion of the N- or C-terminus of an A44 or K39 peptide. In certain embodiments, the fragment is an N- or C-terminal deletion or truncation of an A44 or K39 peptide. In certain embodiments, the fragment of any of the foregoing minimal lengths includes the amino acid sequence: PNPNPNPN. In certain embodiments, the fragment of any of the foregoing minimal lengths and/or the conjugate includes the amino acid sequence: PNPNPNPNP or PNPNPNPNPNPN or PNPNPNPNPNPNP.

In certain embodiments, the A44 or K39 peptide (e.g., the A44/K39 peptide portion) comprises a variant A44 or K39 peptide sequence differing by less than or equal to 20% versus the native sequence. In other words, in certain embodiments, the conjugate comprises an A44 or K39 peptide comprising an amino acid sequence at least 80%, 85%, 90%, 91%, 92%, 93%, 94%, 95%, 96%, 97%, 98%, 99%, or 100% identical to the A44 or K39 peptide sequence set forth in SEQ ID NO: 1 or SEQ ID NO: 2, or a functional fragment thereof. In certain embodiments, the peptide or fragment is capable of entering a cell by endocytosis and translocating to mitochondria. In certain embodiments, the variant or functional fragment retains one or more functional characteristics of native, full length A44 or K39, such as a characteristic selected from one or more of: capable of entering epithelial cells and, optionally, other cell types; capable of entering epithelial cells and, optionally other cell types by endocytosis; capable of translocating to mitochondria; capable of transitting at least one mitochondrial membrane; capable of transitting both mitochondrial membranes; capable of promoting or increasing expression of one or more anti-apoptotic gene (e.g., bcl-2, bcl-XL, NFkB, and mcl-1): capable of decreasing or promoting decrease in expression of one or more pro-apoptotic genes (e.g., bax, bak and TNFa); capable of increasing or promoting increase in expression of one or more pro-survival genes (e.g., rhoA, stat3, and cIAP1). In certain embodiments, a suitable functional fragment or variant retains one or more of the foregoing functions but does not possess one or more other functions. For example, in certain embodiments, it may be desirable to use a fragment or variant of the A44 or K39 peptide that enters cells and translocates to mitochondria but does not increase expression of one or more anti-apoptotic genes. In certain embodiments, a suitable functional fragment or variant retains two or more of the foregoing functions, such as the ability to enter cells by endocytosis and the ability to translocate to mitochondria. In certain embodiments, the functional fragment or variant has at least one, two, three, four, or five, or even all of the foregoing characteristics. These characteristics may be evaluated in a suitable cell based assay. Here, and throughout, these functional features may be used to describe the A44/K39 peptide portion and/or to describe the conjugate.

In certain embodiments, neither the A44/K39 peptide portion, nor the conjugate comprise the amino acid sequence set forth in SEQ ID NO: 1 and/or SEQ ID NO: 2. In other words, the conjugate may comprise e.g. a portion of the A44 or K39 peptide but not the full length peptide.

In certain embodiments, the conjugate comprises a gingipain adhesin peptide comprising an amino acid sequence at least 80%, 85%, 90%, 92%, 95%, 96%, 97%, 98%, 99%, or even 100% identical to SEQ ID NO: 1, or a fragment thereof capable of entering a cell by endocytosis and translocating to mitochondria. In other words, the A44/K39 peptide portion comprises or consists of one of the foregoing. In certain embodiments, the conjugate comprises a gingipain adhesin peptide comprising an amino acid sequence at least 80% identical to SEQ ID NO: 1, or a fragment thereof capable of entering a cell by endocytosis and translocating to mitochondria. In a certain embodiments, the conjugate comprises a gingipain adhesin peptide comprising an amino acid sequence at least 90% identical to SEQ ID NO: 1, or a fragment thereof capable of entering a cell by endocytosis and translocating to mitochondria. In a certain embodiments, the conjugate comprises a gingipain adhesin peptide comprising an amino acid sequence at least 95% identical to SEQ ID NO: 1, or a fragment thereof capable of entering a cell by endocytosis and translocating to mitochondria. In certain embodiments of any of the foregoing, the variant A44 peptide includes the amino acid sequence: PNPNPNPN, PNPNPNPNP or PNPNPNPNPNPN or PNPNPNPNPNPNP. In certain embodiments, the relevant fragment is a fragment of any of the minimal lengths specified herein.

In certain embodiments, the conjugate comprises a gingipain adhesin peptide comprising an amino acid sequence at least 80%, 85%, 90%, 92%, 95%, 96%, 97%, 98%, 99%, or even 100% identical to SEQ ID NO: 2, or a fragment thereof capable of entering a cell by endocytosis and translocating to mitochondria. In other words, the A44/K39 peptide portion comprises or consists of one of the foregoing. In certain embodiments, the conjugate comprises a gingipain adhesin peptide comprising an amino acid sequence at least 80% identical to SEQ ID NO: 2, or a fragment thereof capable of entering a cell by endocytosis and translocating to mitochondria. In certain embodiments, the conjugate comprises a gingipain adhesin peptide comprising an amino acid sequence at least 90% identical to SEQ ID NO: 2, or a fragment thereof capable of entering a cell by endocytosis and translocating to mitochondria. In certain embodiments, the conjugate comprises a gingipain adhesin peptide comprising an amino acid sequence at least 95% identical to SEQ ID NO: 2, or a fragment thereof capable of entering a cell by endocytosis and translocating to mitochondria. In certain embodiments of any of the foregoing, the variant K39 peptide includes the amino acid sequence: PNPNPNPN, PNPNPNPNP or PNPNPNPNPNPN or PNPNPNPNPNPNP. In certain embodiments, the relevant fragment is a fragment of any of the minimal lengths specified herein.

In certain embodiments, the conjugate comprises a gingipain adhesin peptide comprising an amino acid sequence at least 80%, 85%, 90%, 92%, 95%, 96%, 97%, 98%, 99%, or even 100% identical to SEQ ID NO: 3 or SEQ ID NO: 4, or a fragment thereof capable of entering a cell by endocytosis and translocating to mitochondria. In other words, the A44/K39 peptide portion comprises or consists of one of the foregoing. In certain embodiments, the conjugate comprises a gingipain adhesin peptide comprising an amino acid sequence at least 80% identical to SEQ ID NO: 3 or SEQ ID NO: 4, or a fragment thereof capable of entering a cell by endocytosis and translocating to mitochondria. In certain embodiments, the conjugate comprises a gingipain adhesin peptide comprising an amino acid sequence at least 90% identical to SEQ ID NO: 3 or SEQ ID NO: 4, or a fragment thereof capable of entering a cell by endocytosis and translocating to mitochondria. In certain embodiments, the conjugate comprises a gingipain adhesin peptide comprising an amino acid sequence at least 95% identical to SEQ ID NO: 3 or SEQ ID NO: 4, or a fragment thereof capable of entering a cell by endocytosis and translocating to mitochondria. In certain embodiments of any of the foregoing, the variant A44 of K39 peptide fragment includes the amino acid sequence: PNPNPNPN, PNPNPNPNP or PNPNPNPNPNPN or PNPNPNPNPNPNP. In certain embodiments, the relevant fragment is a fragment of any of the minimal lengths specified herein.

In certain embodiments, the conjugate comprises a gingipain adhesin peptide comprising an amino acid sequence at least 80%, 85%, 90%, 92%, 95%, 96%, 97%, 98%, 99%, or even 100% identical to SEQ ID NO: 5 or SEQ ID NO: 6, or a fragment thereof capable of entering a cell by endocytosis and/or translocating to mitochondria. In other words, the A44/K39 peptide portion comprises or consists of one of the foregoing. In certain embodiments, the conjugate comprises a gingipain adhesin peptide comprising an amino acid sequence at least 80% identical to SEQ ID NO: 5 or SEQ ID NO: 6, or a fragment thereof capable of entering a cell by endocytosis and/or translocating to mitochondria. In certain embodiments, the conjugate comprises a gingipain adhesin peptide comprising an amino acid sequence at least 90% identical to SEQ ID NO: 5 or SEQ ID NO: 6, or a fragment thereof capable of entering a cell by endocytosis and/or translocating to mitochondria. In certain embodiments, the conjugate comprises a gingipain adhesin peptide comprising an amino acid sequence at least 95% identical to SEQ ID NO: 5 or SEQ ID NO: 6, or a fragment thereof capable of entering a cell by endocytosis and translocating to mitochondria. In certain embodiments of any of the foregoing, the variant A44 or K39 peptide fragment includes the amino acid sequence: PNPNPNPN, PNPNPNPNP or PNPNPNPNPNPN or PNPNPNPNPNPNP. In certain embodiments, the relevant fragment is a fragment of any of the minimal lengths specified herein.

In certain embodiments, the A44/K39 peptide portion is capable of promoting entry across at least one mitochondrial membrane. In certain embodiments, the A44/K39 peptide portion is capable of promoting entry across both mitochondrial membranes. In either case, in certain embodiments, a conjugate comprising such A44/K39 peptide portion is capable of transiting across one or, in certain embodiments, both mitochondrial membranes. In certain embodiments, of any of the foregoing or following, the fragment includes the N-terminus of an A44 or K39 peptide.

In certain embodiments, the A44/K39 peptide portion comprises one or more of the following functional characteristics: capable of entering epithelial cells and, optionally, other cell types; capable of entering epithelial cells and, optionally other cell types by endocytosis; capable of translocating to mitochondria; capable of transitting at least one mitochondrial membrane; capable of transitting both mitochondrial membranes; capable of promoting or increasing expression of one or more anti-apoptotic gene (e.g., bcl-2, bcl-XL, NFkB, and mcl-1); capable of decreasing or promoting decrease in expression of one or more pro-apoptotic genes (e.g., bax, bak and TNFa): capable of increasing or promoting increase in expression of one or more pro-survival genes (e.g., rhoA, stat3, and cIAP1). In certain embodiments, the conjugate retains such one or more functional characteristics and can be described based on such one or more characteristics. Any conjugate of the disclosure can be described based on the structural and/or functional characteristics of the A44/K39 peptide portion and/or the heterologous agent portion. In certain embodiments, a suitable functional fragment or variant (or any A44/K39 peptide portion) retains one or more of then foregoing functions but does not possess one or more other functions. For example, in certain embodiments, it may be desirable to use a fragment or variant of the A44 or K39 peptide that enters cells and translocates to mitochondria but does not increase expression of one or more anti-apoptotic genes. In certain embodiments, a suitable functional fragment or variant retains two or more of the foregoing functions, such as the ability to enter cells by endocytosis and the ability to translocate to mitochondria. In certain embodiments, the functional fragment or variant has at least one, two, three, four, or five, or even all of the foregoing characteristics. These characteristics may be evaluated in a suitable cell based assay.

In certain embodiments, endocytosis is clathrin-mediated endocytosis. In certain embodiments, an A44/K39 peptide portion of the disclosure and/or conjugate of the disclosure is capable of entering a cell, such as an epithelial cell, via clathrin-mediated endocytosis.

In certain embodiments, the A44/K39 peptide portion is substantially non-toxic. In other words, the A44/K39 peptide portion may be administered to cells in culture at a relevant dose without significant toxicity, as measured in vitro. An exemplary dose is a dose sufficient to observe some positive effect without significant toxicity.

The disclosure also provides conjugates comprising various types of heterologous agents. When present as part of a conjugate, the heterologous agent may be transported along with the A44/K39 peptide portion into cells and to the mitochondria. In certain embodiments, the heterologous agent is a protein, peptide, polynucleotide, oligonucleotide or small organic or inorganic molecule. In certain embodiments, the heterologous agent is a polypeptide or peptide, and wherein the polypeptide or peptide is selected from: an enzyme, a cofactor, a cytochrome, an antibody or a growth factor. We note that the terms polypeptide and peptide are used interchangeably, unless context indicates otherwise.

In certain embodiments, the heterologous agent is a pharmaceutical agent. In certain embodiments, the heterologous agent is a nutraceutical agent, for example vitamin E or vitamin C. In certain embodiments, the heterologous agent is a vitamin. In certain embodiments, the heterologous agent is an antioxidant. In certain embodiments, the heterologous agent is a therapeutic agent or a therapeutically active agent. It is appreciated that these categories of agents may, in certain embodiments, be overlapping. In certain embodiments, the heterologous agent is endogenously present or active in mitochondria. In certain embodiments, the heterologous agent is suitable for treating a mitochondrial disease or condition. The selection of an appropriate heterologous agent is dependent on the desired therapeutic/biological effect sought in a particular cell or subject.

The disclosure also provides for multiple configurations of the A44 or K39 peptide, or fragment thereof relative to the heterologous agents (e.g., multiple configurations of the A44/K39 peptide portion vis-à-vis the heterologous agent portion). In other words, conjugates of various orientations and configurations are contemplated and provided herein. In certain embodiments, the heterologous agent is N-terminal to the peptide, such as linked (directly or indirectly) via the N-terminal amino acid of the A44/K39 peptide portion. In certain embodiments, the heterologous agent is C-terminal to the peptide. In certain embodiments, the heterologous agent is linked, directly or indirectly, to the C-terminal amino acid of the A44/K39 peptide portion. In other embodiments, the heterologous agent is linked, directly or indirectly, to an internal amino acid of the A44/K39 peptide portion.

In certain embodiments, the conjugate further comprises a linker interconnecting the A44/K39 peptide portion and the heterologous agent. In certain embodiments, the A44/K39 peptide portion and heterologous agent are directly interconnected without a linker. In certain embodiments, A44/K39 peptide portion and heterologous agent are indirectly interconnected with a linker, which separates the peptide and the heterologous agent by a distance sufficient to ensure that each component retains its proper structure and functional capabilities and/or allow for a flexible extended conformation to promote, e.g. interaction with additional components. One class of linkers is glycine-serine linkers.

The disclosure also provides for multiple types of attachments or associations of the A44 or K39 peptide, or fragment thereof to the heterologous agents. In other words, conjugates of the disclosure include conjugates in which the A44/K39 peptide portion and heterologous agent portion are associated in numerous different ways, including covalently. Moreover, the A44/K39 peptide portion and heterologous agents may be covalently associated via a peptide bound, such as by forming a fusion protein, or via a chemical conjugation. In certain embodiments, the peptide and the heterologous agent are chemically conjugated. The term conjugate is used to encompass any such association and the appropriate association and configuration is selected based on the A44/K39 peptide portion and heterologous agent.

In certain embodiments, the conjugate comprises a fusion protein comprising the polypeptide and the heterologous agent. In certain embodiments, the fusion protein is produced using a recombinant vector encoding both the polypeptide and the heterologous agent. In related embodiments, the recombinant vector further comprises a peptide linker between the polypeptide and the heterologous agent.

The present disclosure also provides for further modification of the conjugate. In certain embodiments, if the conjugate comprises an epitope tag it further comprises an additional heterologous agent. In other words, an epitope tag (e.g., His-tag or myc-tag, the purpose of with is solely to facilitate protein purification or detection but which does not have an independent function as a heterologous agent) is not itself a heterologous agent but may be present as part of a conjugate of the disclosure.

In certain embodiments, the heterologous agent is or the conjugate further comprises a moiety to increase in vivo stability or in vivo half life, e.g. a nonproteinaceous polymer, such as polyethylene glycol, polypropylene glycol, or polyoxyalkylenes. By way of example, a native A44 or K39 peptide can increase expression or activity of one or more pro-survival factors and/or decrease expression or activity of one or more pro-apoptotic factors. Accordingly, delivery of A44 or K39 conjugated to a nonproteinaceous polymer to extent half life and/or stability is itself useful therapeutically and as a useful reagent. Similarly, further appending a conjugate of the disclosure has similar benefits. Pegylation may also be useful for improving safety and efficacy because, for example, conjugates or A44/K39 peptide portions with greater stability or half-life can be administered at a lower dose or less frequently.

In certain embodiments, the conjugate further comprises a moiety to increase detection, uptake/administration, production, or purification, e.g., polypeptides that include epitope tags, such as HA and myc tags, the Fc region of an immunoglobulin or all or a portion of HSA. In certain embodiments, the conjugate further comprises an Fc portion of an antibody.

The disclosure provides conjugates comprising an A44 or K39 peptide, or fragment or variant thereof (e.g., A44/K39 peptide portion) that is capable of entering a variety of cell types by endocytosis and translocating to mitochondria. In certain embodiments, the cell (e.g., the cell into which the conjugate is capable of entering) is an endodermal-derived cell, such as a pancreatic cell, a lung cell, or a thyroid cell. In certain embodiments, the cell is a mesodermal-derived cell, such as a fibroblast, an endothelial cell, a myocyte, hepatocyte, or an adipocyte. In certain embodiments, the cell is an ectodermal-derived cell, such as an epithelial cell, a neuronal cell or a glial cell. In certain embodiments, the cell is an epithelial cell.

The disclosure provides compositions comprising a conjugate disclosed herein and one or more pharmaceutically acceptable carriers and/or excipients. In certain embodiments, the conjugates for use in these compositions are substantially purified or pyrogen free.

In certain embodiments, conjugates of the disclosure are isolated and/or substantially purified. In certain embodiments, a conjugate of the disclosure is greater than 80% by weight (at least 81%, 85%, 90%, 91%, 92%, 93%, 94%, 95%, 96%, 97%, 98%, or at least 99%) of the active ingredient in a composition. For example, in a composition, in certain embodiments, greater than 80% of the active ingredient in the composition is conjugate of the disclosure. The compositions may further include one or more pharmaceutically acceptable carriers and/or excipients. In certain embodiments, by purified means that greater than 80% by weight of the species present in the composition is conjugate of the disclosure (at least 81%, 85%, 90%, 91%, 92%, 93%. 94%, 95%, 96%, 97%, 98%, or at least 99%).

The disclosure provides nucleic acid constructs comprising a nucleotide sequence that encodes (i) a conjugate described herein or (ii) the peptide portion of a conjugate described herein, wherein, for example, the nucleic acid construct encodes a conjugate capable of entering a cell by endocytosis and localizing to mitochondria and/or retaining one or more functions of native, full length A44 or K39 peptide. The disclosure also provides host cells comprising the nucleic acid described herein. The disclosure further provides methods of producing the conjugate described herein, comprising culturing the host cell described herein under conditions for producing said conjugate and its. In certain embodiments, the method further comprising isolating said conjugate. In certain embodiments, the method further comprises purifying the conjugate such that it is pyrogen free.

The disclosure provides for a method for treating a mitochondrial disorder or mitochondrial dysfunction in a cell, the method comprising administering effective amount of a conjugate disclosed herein. Similarly the disclosure provides methods for decreasing one or more symptoms or effects of mitochondrial dysfunction in a cell.

The disclosure provides for a method for delivering a heterologous agent to mitochondria in a cell, the method comprising administering an effective amount of a conjugate of the disclosure, such as a conjugate comprising any A44/K39 peptide portion and any heterologous agent portion. In certain embodiments, the method comprises an in vivo method and an effective amount of conjugate is administered to a patient. In certain embodiments, the method comprises an in vitro method. In certain embodiments, the method comprises delivering the heterologous agent across one or both mitochondrial membranes. In certain embodiments, the method comprises delivering the heterologous agent across both mitochondrial membranes.

In certain embodiments, the conjugate is capable of penetrating at least one mitochondrial membrane. In certain embodiments, the conjugate is capable of penetrating both mitochondrial membranes. Accordingly, in certain embodiments, the disclosure provides a method of delivering a heterologous agent across one or both mitochondrial membranes, thereby delivering the heterologous agent into mitochondria in a cell.

The disclosure provides a method of preventing or decreasing cell death in a cell, the method comprising administering an effective amount of a conjugate of the disclosure.

The disclosure provides a method of preventing or decreasing oxidative stress in a cell comprises administering an effective amount of a conjugate of the disclosure.

The disclosure provides a method of reducing the number of mitochondria undergoing mitochondrial permeability transitioning (MPT) or preventing or decreasing mitochondrial permeability transitioning in a cell, the method comprising administering an effective amount of a conjugate of the disclosure.

The disclosure provides a method for delivering an agent into a cell and to mitochondria in a cell, comprising contacting a cell with an effective amount of a conjugate of the disclosure.

The disclosure provides a method of preventing or decreasing oxidative stress in a cell, comprising administering an effective amount of a conjugate of the disclosure.

The disclosure provides a method of decreasing reactive oxygen species in a cell, comprising administering an effective amount of a conjugate of the disclosure

In certain embodiments, the method is an in vitro method, and administering an effective amount of a conjugate comprises contacting cells with a conjugate, such as by adding the conjugate to culture media. In certain embodiments, the cells in vitro are epithelial cells. In certain embodiments, the method is an in vivo method, and administering an effective amount of a conjugate comprises administering conjugate to a subject, such as a subject in need thereof (e.g., having mitochondrial dysfunction). In certain embodiments, the cell (either in vitro or in vivo) is characterized by mitochondrial dysfunction.

The disclosure provides a method of detecting mitochondrial dysfunction in a cell in a subject in need thereof, the method comprising administering to the subject an effective amount of a conjugate of the disclosure.

The disclosure provides for a method of detecting cell death in a cell in a subject in need thereof, the method comprising administering to the subject an effective amount of a conjugate of the disclosure.

The disclosure provides for a method of detecting oxidative stress in a cell in a subject in need thereof, the method comprising administering to the subject an effective amount of a conjugate of the disclosure.

The disclosure provides a method of predicting an effect of a conjugate or heterologous agent on a cell in vitro or in vivo, the method comprising i) obtaining a cell from a subject; ii) exposing the cell obtained from the subject to a conjugate of the disclosure; and iii) assaying for a pharmacological or toxicological effect of the conjugate on the cell relative to a control cell not exposed to the conjugate or exposed to heterologous agent that is not associated with a A44/K39 peptide portion (or a cell treated with either portion of the conjugate alone). In certain embodiments of the methods, the pharmacological effect is a change in mitochondrial activity or mitochondrial gene expression.

The disclosure provides a method of identifying an effect of a conjugate of a heterologous agent, the method comprising exposing a cell to a conjugate of the disclosure and assaying for a pharmacological or toxicological effect of the conjugate on the cell relative to a control cell not exposed to the conjugate or exposed to heterologous agent that is not associated with a A44/K39 peptide portion (or a cell treated with either portion of the conjugate alone). In certain embodiments of the methods, the pharmacological effect is a change in mitochondrial activity or mitochondrial gene expression or a change in presence of reactive oxygen species. In certain embodiments, this method is used to evaluate an improvement in the activity of a heterologous agent when present as a conjugate associated with an A44/K39 peptide portion and targeted to mitochondria.

In certain embodiments, the A44/K39 peptide portion alone is not toxic to cells and that level of toxicity (or lack of toxicity) is compared to that of the heterologous agent alone and/or to the conjugate.

In certain embodiments of the methods, administering the conjugate results in increased expression of one or more anti-apoptotic genes, e.g., bcl-2, bcl-XL, NFkB, and mcl-1, relative to an untreated cell (or a cell treated with either portion of the conjugate alone). In certain embodiments of the methods, administering the conjugate results in decreased expression of one or more pro-apoptotic genes, e.g., bax, bak and TNFa, relative to an untreated cell (or a cell treated with either portion of the conjugate alone). In certain embodiments of the methods, administering the conjugate results in increased expression of one or more pro-survival genes, e.g., rhoA, stat3, and cIAP1, relative to an untreated cell (or a cell treated with either portion of the conjugate alone). These properties are also suitable for describing or defining fragments or variants suitable for use as an A44/K39 peptide portion. Note, however, in other embodiments, the conjugate and/or A44/K39 peptide portion does not have one or more of the foregoing affects on target cell gene expression. The disclosure provides a variety of cell types applicable for the methods disclosed herein. In certain embodiments, the cell is an endodermal-derived cell, such as a pancreatic cell, a lung cell, or a thyroid cell. In certain embodiments, the cell is a mesodermal-derived cell, such as a fibroblast, an endothelial cell, a myocyte, hepatocyte, or an adipocyte. In certain embodiments, the cell is an ectodermal-derived cell, such as an epithelial cell, a neuronal cell or a glial cell. In certain embodiments, the cell is an epithelial cell. In another preferred embodiment, the cell is a human cell.

In certain embodiments of the methods, the cell is characterized by a mitochondrial dysfunction and/or is a cell from or of a subject having a mitochondrial disorder or condition.

In certain embodiments of the methods, the cell is in a subject. In certain embodiments of the methods, the subject is a subject in need of treatment for a mitochondrial disease or condition. In certain related embodiments of the methods, the disease or condition is characterized by increased oxidative damage in a cell and/or increased cell death, for example a disease or condition characterized by degeneration e.g., cardiovascular diseases such as atherosclerosis, ischemia/reperfusion injury, heart failure, stroke, and traumatic brain injury; neurodegenerative diseases such as Alzheimer's disease, Parkinson's disease, Huntington's disease, amyotrophic lateral sclerosis (ALS), autism, and muscular dystrophy; chronic autoimmune inflammatory diseases such as rheumatoid arthritis; and metabolic diseases such as diabetes and obesity. In certain embodiments of the methods, wherein the disease or condition is characterized by increased cell death, the conjugate comprises a heterologous agent that promotes cell survival and/or uses an A44/K39 peptide portion that increases expression of one or more pro-survival genes and/or decreases expression of one or more pro-apoptotic genes.

In certain embodiments of the methods, the disease or condition is characterized by a hyperproliferative state, e.g., cancer. In certain embodiments of the methods, wherein the disease or condition is characterized by hyperproliferation, the conjugate comprises a heterologous agent that promotes cell death or is cytotoxic. In certain embodiments, when conjugates are selected, made and used in this context, an A44/K39 peptide portion that does not retain certain pro-survival and/or anti-apoptotic properties of native A44 or K39 may be selected and used.

In certain embodiments of the methods, the conjugate is administered orally, topically, intranasally, systemically, intravenously, subcutaneously, intramuscularly, or transdermally. Moreover, in certain embodiments, a composition is formulated for oral, topical, intranasal, systemic, intravenous, subcutaneous, intramuscular or transdermal administration (e.g., the composition comprises an intravenous formulation).

The disclosure contemplates combinations of any of the features, aspects and embodiments of the disclosure. For example, the disclosure contemplates conjugates comprising any A44/K39 peptide portion and any heterologous agent portion. These portions and the conjugates can be described using any combination of structural and/or functional properties disclosed herein. For example, A44/K39 peptide portions having one or more functional features (e.g., 1, 2, 3, 4, 5 or more than 5) of a native A44 or K39 peptide may be selected (e.g., A44/K39 peptide portions), and any such A44/K39 peptide portions may be associated with a heterologous agent. Any such conjugates of the disclosure may be used, for example, in any of the in vitro or in vivo methods described herein.

### BRIEF DESCRIPTION OF THE FIGURES

FIG. 1 provides a schematic representation showing the topology of RgpA (top) and Kgp (bottom) gingipain proteins. FIG. 1 is a schematic representation showing the catalytic domain and four adhesin domains of RgpA and Kgp gingipain proteins. Same shading indicates regions sharing near 100% amino acid identity. The various adhesin peptides of RgpA and Kgp are shown in FIG. 1, including the A44 and K39 peptides.

FIG. 2 is an amino acid sequence alignment between the native full length A44 peptide from RgpA and the native, full length K39 peptide from Kgp. The amino acid sequence of this native, full length A44 peptide is set forth in SEQ ID NO: 1 and the amino acid sequence of this native, full length K39 peptide are set forth in SEQ ID NO: 2. Nucleotide sequence encoding a native A44 or K39 peptide can also be amplified from chromosomal DNA of P. gingivalis (ATCC 33277). Native A44 peptide and native K39 peptide amplified from such chromosomal DNA is also included within the scope of A44 and K39 peptides and any peptides of the disclosure can be similarly so described.

FIG. 3 is a schematic representation summarizing the ability of exogenous provided A44-based peptides to be taken up by cells and translocated to the mitochondria. A schematic representation of each of the tested A44 peptide portions appear on the left and are as follows, from top down: A44 peptide (full-length) (SEQ ID NO: 1), A44N4C4 (a fragment of the A44 peptide; SEQ ID NO: 11), A44N1 (a fragment of the A44 peptide; SEQ ID NO: 19), A44N2 (a fragment of the A44 peptide; SEQ ID NO: 25), A44C2 (a fragment of the A44 peptide; SEQ ID NO: 23), A44C1 (a fragment of the A44 peptide; SEQ ID NO: 13), A44N2C5 (a fragment of the A44 peptide; SEQ ID NO: 21), A44NC5 (a fragment of the A44 peptide; SEQ ID NO: 7), A44N2C4 (a fragment of the A44 peptide; SEQ ID NO.: 5), A44NC4 (a fragment of the A44 peptide; SEQ ID NO: 3), A44N3C5 (a fragment of the A44 peptide; SEQ ID NO: 17), A44N3C4 (a fragment of the A44 peptide; SEQ ID NO: 15) and A44repdel (a fragment of the A44 peptide; SEQ ID NO: 9). Also shown is a schematic representation of the K39 peptide (full-length) (SEQ ID NO: 2) in the last row. Peptides for which attachment to the cell was observed are indicated by a “+” in the column labeled “attached”. Peptides for which such attachment was not observed are indicated by a “−” in the column labeled “attached”. Peptides capable of cellular uptake are indicated by a “+” in the column labeled “uptake”. Peptides for which cellular uptake was not observed are indicated by a “−” in the column labeled “uptake”. Peptides capable of cellular uptake followed by translocation to the mitochondria are indicated by a “+” in the column labeled “mitochondria translocation”. Peptides for which cellular uptake followed by translocation to the mitochondria was not observed are indicated by a “−” in the column labeled “mitochondria translocation”. Lanes marked as “tbd” have not yet been tested.

FIG. 4A-C presents images of Western Blots of mitochondrial fractions of human epithelial type 2 (HEp-2) cells (FIG. 4A), oral keratinocytes (Oba-9) (FIG. 4B) and mouse fibroblasts (NIH 3T3) (FIG. 4C) probed with an anti-his-tag antibody (HRP-labeled secondary) to detect the recombinant A44 peptide.

FIG. 5A-B are images of a 3D gingival tissue stained with an anti-his-tag antibody to detect the recombinant A44 peptide (brown) and counterstained with hematoxylin to visualize nuclei (blue).

### DETAILED DESCRIPTION

### I. Overview

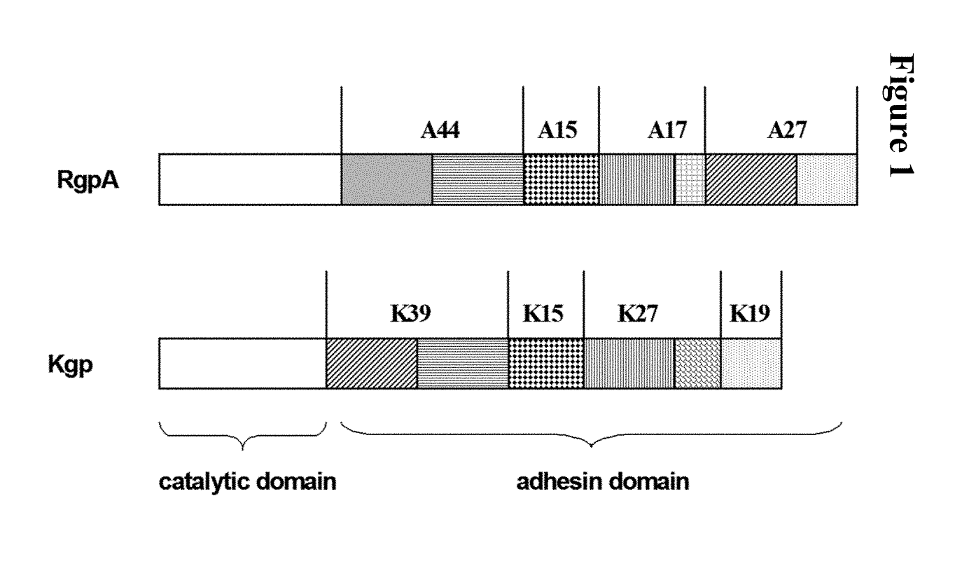

Gingipains are a family of cysteine proteases produced by the Gram-negative oral anaerobe Porphyromonas gingivalis, a major cause of periodontal disease that has also been associated with other major systemic diseases (Rosenstein E D, Greenwald R A, Kushner L J, Weissmann G (2004) Hypothesis: The humoral immune response to oral bacteria provides a stimulus for the development of rheumatoid arthritis. Inflammation 28(6):311-318). Gingipains are found on the surface of cells, in extracellular vesicles, and in culture supernatants. Gingipains, which include Arg-gingipain (Rgp) and Lys-gingipain (Kgp), are composed of an N-terminal catalytic domain and a series of C-terminal adhesin domains. During growth of P. gingivalis, the N-terminal catalytic domain autoprocesses the C-terminal adhesin domains into smaller adhesin peptides. Autoprocessing of the adhesin domains of RgpA yields the A44, A15, A17 and A27 peptides (FIG. 1). Autoprocessing of the adhesin domains of Kgp yields the K39, K15, K27 and K19 peptides (FIG. 1). The amino acid sequence of the native, full length A44 peptide of Rgp is set forth in SEQ ID NO: 1. The amino acid sequence of the native, full length K39 peptide of Kgp is set forth in SEQ ID NO.: 2. Moreover, the amino acid sequence of the full length Arg-gingipain (Rgp) protein is set forth in SEQ ID NO: 27 and the amino acid sequence of the full length Lys-gingipain (Kgp) protein is set forth in SEQ ID NO: 28.

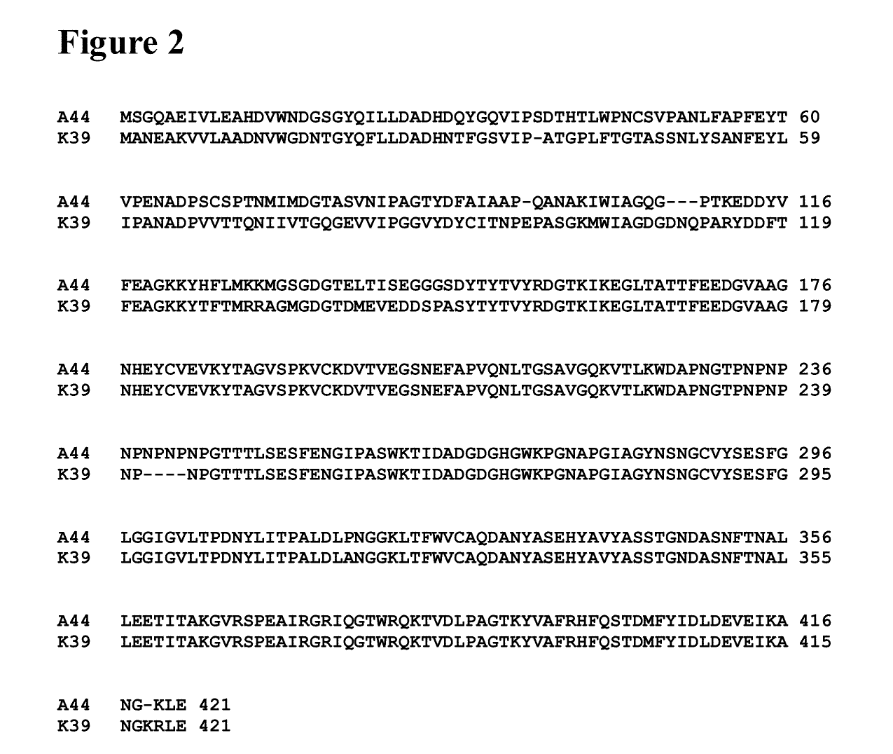

Gingipains interact with host cells to mediate entry of P. gingivalis into a host cell (Boisvert and Duncan, Cellular Microbiology, 10(12):2538-52 (2008)) and, following entry, translocation of P. gingivalis to mitochondria (Boisvert and Duncan, Infection and Immunity, 78(8): 3616-3624 (2010)). The full length A44 peptide, which shares significant homology with the K39 peptide (see FIGS. 1 and 2), is capable of being endocytosed via clathrin-dependent pathway into a host epithelial cell and, following entry is not degraded. Rather, the full length A44 peptide subsequently translocates to mitochondria, where it can be found within the mitochondrial organelle.

The present disclosure provides conjugates comprising (i) an A44/K39 peptide portion and (ii) a heterologous agent portion associated therewith. In certain embodiments, the association is covalent (e.g., the portions are interconnected, directly or via a linker, covalently). In certain embodiments, the conjugate and/or A44/K39 peptide portion is capable of entering cells, such as epithelial cells, and translocating to mitochondria. In certain embodiments, the conjugate and/or A44/K39 peptide portion have one or more functions of native, full length A44 or K39 peptide, such as one or more functions selected from: capable of entering epithelial cells and, optionally, other cell types; capable of entering epithelial cells and, optionally, other cell types by endocytosis; capable of translocating to mitochondria; capable of transitting at least one mitochondrial membrane; capable of transitting both mitochondrial membranes; capable of promoting or increasing expression of one or more anti-apoptotic gene (e.g., bcl-2, bcl-XL, NFkB, and mcl-1); capable of decreasing or promoting decrease in expression of one or more pro-apoptotic genes (e.g., bax, bak and TNFa): capable of increasing or promoting increase in expression of one or more pro-survival genes (e.g., rhoA, stat3, and cIAP1). Suitable A44/K39 peptide portions comprise: full length A44 or K39, functional fragments of native A44 or K39; variants thereof having at least 80% amino acid sequence identity to the corresponding portion of native A44 or K39 and that retain the specified function. Exemplary suitable A44/K39 peptides portions are described herein. In certain embodiments, the A44/K39 peptide portion is selected so that it has one or more functions (e.g., 1, 2, 3, 4, 5, or more than 5) of native A44 or K39 peptide but does not have one or more other functions. For example, in certain embodiments, the A44/K39 peptide portion is capable of entering cells via endocytosis and transolocating to mitochondria, but does not increase expression of one or more pro-survival factors. In other words, in certain embodiments, the A44/K39 peptide portion serves as a useful carrier/delivery compound but does not itself alter certain gene expression. In certain embodiments, the A44/K39 peptide portion and/or conjugate are non-toxic or substantially non-toxic.

Conjugates of the disclosure are suitable, for example, for facilitating delivery of heterologous agents into cells, such as epithelial cells, and to mitochondria, such as into mitochondria. Accordingly, conjugates of the disclosure have numerous in vitro and in vivo uses.

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “conjugate of the disclosure” is used to refer to a complex comprising an A44/K39 peptide portion associated with a heterologous agent, such as a polypeptide, peptide, polynucleotide, oligonucleotide or small molecule. In certain embodiments, the complex comprises a covalent interconnection associating these two portions. The term conjugate is used to refer to these complexes regardless of orientation and the nature of the interconnection (e.g., it includes conjugates in which the interconnection is a chemical linkage as well as a peptide bond, such as to form a fusion protein).

The term “A44/K39 peptide portion” is used to refer to full length native A44 or K39 peptide, or functional fragments thereof or variants thereof having at least 80% amino acid identity. In other words, an A44/K39 peptide portion corresponds to a polypeptide comprising or consisting of an A44 or K39 peptide, or a functional fragment thereof or a variant thereof having at least 80% amino acid identity, as described herein. In certain embodiments, A44/K39 peptide portions for use in the conjugates of the disclosure retain at least the following functions of full length A44: the ability to enter, at least, epithelial cells and the ability to traffic or translocate to mitochondria. In certain embodiments, A44/K39 peptide portions for use in the conjugates of the disclosure retain one or more of the following functions of native, full length A44: capable of entering epithelial cells and, optionally, other cell types; capable of entering epithelial cells and, optionally other cell types by endocytosis; capable of translocating to mitochondria; capable of transitting at least one mitochondrial membrane; capable of transitting both mitochondrial membranes; capable of promoting or increasing expression of one or more anti-apoptotic gene (e.g., bcl-2, bcl-XL, NFkB, and mcl-1); capable of decreasing or promoting decrease in expression of one or more pro-apoptotic genes (e.g., bax, bak and TNFa); capable of increasing or promoting increase in expression of one or more pro-survival genes (e.g., rhoA, stat3, and cIAP1). In certain embodiments, an A44/K39 peptide portion retains one or more functions of full length, native A44 peptide (or K39 peptide)

Unless context indicates otherwise, the term “A44 peptide” or “A44 gingipain adhesion peptide” refers to a full length A44 peptide, such as a full length A44 peptide from or having a sequence corresponding to that of the A44 peptide of Arg-gingipain (Rgp) protein.

Unless context indicates otherwise, the term “K39 peptide” or “K39 gingipain adhesion peptide” refers to a full length K39 peptide, such as a full length K39 peptide from or having a sequence corresponding to that of the K39 peptide of Lys-gingipain (Kgp) protein.

### II. A44 and K39 Peptides

Presently disclosed are full length A44 and K39 peptides and fragments or variants thereof (e.g., collectively A44/K39 peptide portions). Conjugates comprising (or consisting of) such peptides and fragments are useful for a variety of purposes. Provided below are examples of particular A44 and K39 peptides and fragments (including variants) and exemplary uses.

The term A44/K39 peptide portion is used to refer to any of the A44 or K39 peptides and fragments or variants thereof. The disclosure provides conjugates comprising an A44/K39 peptide portion and a heterologous agent portion. Such conjugates are suitable for a variety of in vitro and in vivo purposes, including therapeutic, diagnostic and research uses. The A44/K39 peptide portion can be described structurally (e.g., by amino acid sequence and/or size) and/or by function. Exemplary functions include, but are not limited to, the ability to enter cells (e.g., by endocytosis) and the ability to translocate to mitochondria. In certain embodiments, the A44/K39 peptide portion is capable of trafficking to and into the mitochondria, and may, in certain embodiments, penetrate one or both mitochondrial membranes. In certain embodiments, the A44/K39 peptide portion is capable of regulating the expression of anti-apoptic genes. Functional attributes can be used to describe the A44/K39 peptide portions and/or the conjugate. In other words, in certain embodiments, any of the functions of the A44/K39 peptide portions can be used to describe that portion or the conjugate.

In certain embodiments, the A44/K39 peptide portion may comprise (or consist of) an amino acid sequence that corresponds to a contiguous amino acid sequence present in a full length, native A44 or K39 peptide. In certain embodiments, the A44/K39 peptide portion may comprise (or consist of) an amino acid sequence that corresponds to a contiguous amino acid sequence present in a full length, native A44 or K39 peptide and also include a portion of non-contiguous amino acid sequence. For example, the A44/K39 peptide portion may comprise a fragment of an A44 or K39 peptide that lacks intervening amino acids that occur in the native peptide between two or more regions of the peptide, resulting in a peptide with sequences juxtaposed that are not typically found juxtaposed in the full length, native A44 or K39 peptide.

The disclosure provides conjugates comprising an A44/K39 peptide portion (e.g., which may be a functional fragment or variant of a full length A44 or K39 peptide). Exemplary peptides are described herein. As disclosed herein, an N- or C-terminal portion of the A44 or K39 peptide is capable of mediating cellular uptake (or cell internalization/entry). As further disclosed herein, a (PN)ₙP repeat domain within the A44 and K39 peptide is capable of mediating translocation to mitochondria, and may include e.g. the following repeats: (PN)₆P, (PN)₅P and (PN)₄P.

In certain embodiments, the A44/K39 peptide portion comprises or consists of at least 243, 242, 241, 240, 235, 230, 225, 220, 215, 210, 205, 200, 195, 190, 185, 180, 175, 170, 165, 160, 155, 150, 145, 140, 135, 130, 125, 120, 115, 110, 105, 100, 90, 80, 70, 60 or 50 amino acid residues of full length, native A44 or K39 (e.g., such as a functional fragment of SEQ ID NO.: 1 or 2.). In certain embodiments, the conjugate comprises a functional fragment (such as any of the foregoing fragments or any fragment described herein) but does not comprise full length, native A44 or K39. In certain embodiments, the A44/K39 peptide comprises at least 243 amino acid residues. In certain embodiments, the A44/K39 peptide comprises at least 242 amino acid residues. In certain embodiments, the A44/K39 peptide portion comprises at least 200 amino acid residues. In certain embodiments, the A44/K39 peptide portion comprises at least 150 amino acid residues. In certain embodiments, the A44/K39 peptide portion comprises at least 100, 90, 80, 70, 60 or 50 amino acid residues. The foregoing fragments may comprise functional fragments (e.g., fragments that retain one or more functions of native, full length A44 or K39 but, optionally, do not retain all of the functions of native, full length A44 or K39).

In certain embodiments, the A44/K39 peptide portion comprises an amino acid sequence at least 80%, 85%, 90%, 92%, 95%, 96%, 97%, 98%, 99%, or even 100% identical to SEQ ID NO.: 1 or SEQ ID NO.: 2, such as to the corresponding portion of SEQ ID NOs: 1 or 2 (if comparing a fragment). In certain embodiments, the A44/K39 peptide portion comprises an amino acid sequence at least 80% identical to SEQ ID NO: 1 or SEQ ID NO: 2. In certain embodiments, the A44/K39 peptide portion comprises an amino acid sequence at least 90% identical to SEQ ID NO: 1 or SEQ ID NO: 2. In certain embodiments, the A44/K39 peptide portion comprises an amino acid sequence at least 95%, 96%, 97%, or 98% identical to SEQ ID NO.: 1 or SEQ ID NO.: 2. In certain embodiments, the A44/K39 peptide portion comprises an amino acid sequence 100% identical to SEQ ID NO.: 1 or SEQ ID NO.: 2. Optionally, an A44/K39 peptide portion of the disclosure or a conjugate of the disclosure may include an N-terminal methionine. In certain embodiments, the A44/K39 peptide portion comprises or consists of a functional fragment of any of the foregoing, such as a functional fragment of at least (or of about) 50, 60, 70, 80, 90, 100, 125, 130, 140, 150, 160, 170, 175, 180, 190, 195, 200, 210, 215, 220, 225, 230, 235, 240, 241, 242 or 243 amino acid residues of any of the foregoing. Similarly, fragments of any of the foregoing of less than or equal to 242 or 243 amino acid residues are specifically contemplated, such as fragments of greater than or equal to 50 amino acid residues but less than or equal to 242 or 243 amino acid residues, optionally further including an N-terminal methionine.

In certain embodiments, the A44/K39 peptide portion comprises (or consists of) an amino acid sequence at least 80%, 85%, 90%, 92%, 95%, 96%, 97%, 98%, 99%, or even 100% identical to SEQ ID NO.: 3 or SEQ ID NO.: 4, such as to the corresponding portion of SEQ ID NOs.: 3 or 4 (if comparing a fragment). In certain embodiments, the A44/K39 peptide portion comprises an amino acid sequence at least 80% identical to SEQ ID NO.: 3 or SEQ ID NO.: 4. In certain embodiments, the A44/K39 peptide portion comprises an amino acid sequence at least 90% identical to SEQ ID NO.: 3 or SEQ ID NO.: 4. In certain embodiments, the A44/K39 peptide portion comprises an amino acid sequence at least 95% identical to SEQ ID NO.: 3 or SEQ ID NO.: 4. In certain embodiments, the A44/K39 peptide portion comprises or consists of an amino acid sequence 100% identical to SEQ ID NO.: 3 or SEQ ID NO: 4.

In certain embodiments, the A44/K39 peptide portion comprises an amino acid sequence at least 80%, 85%, 90%, 92%, 95%, 96%, 97%, 98%, 99%, or even 100% identical to SEQ ID NO.: 3; SEQ ID NO.: 4, SEQ ID NO.: 7, SEQ ID NO.: 8, SEQ ID NO.: 9, or SEQ ID NO.: 10, such as to the corresponding portion thereof (if comparing a fragment). In certain embodiments, the A44/K39 peptide portion comprises an amino acid sequence at least 80% identical to SEQ ID NO.: 3; SEQ ID NO.: 4, SEQ ID NO.: 7, SEQ ID NO.: 8, SEQ ID NO.: 9, or SEQ ID NO.: 10. In certain embodiments, the A44/K39 peptide portion comprises an amino acid sequence at least 90% identical to SEQ ID NO.: 3; SEQ ID NO.: 4, SEQ ID NO.: 7, SEQ ID NO.: 8, SEQ ID NO.: 9, or SEQ ID NO.: 10. In certain embodiments, the A44/K39 peptide portion comprises an amino acid sequence at least 95% identical to SEQ ID NO.: 3; SEQ ID NO.: 4, SEQ ID NO.: 7, SEQ ID NO.: 8, SEQ ID NO.: 9, or SEQ ID NO.: 10. In certain embodiments, the A44/K39 peptide portion comprises an amino acid sequence 100% identical to SEQ ID NO.: 3; SEQ ID NO.: 4, SEQ ID NO.: 7, SEQ ID NO.: 8, SEQ ID NO.: 9, or SEQ ID NO.: 10.

For any of the foregoing, it is contemplated that the appropriate fragment or variant suitable for use in a conjugate of the disclosure is a functional fragment or variant, e.g., a fragment or variant that retains one or more functions. In certain embodiments, the A44/K39 peptide portion comprises a functional fragment or variant of native A44 or K39 and is capable of entering cells, such as epithelial cells and such as by endocytosis, and promoting translocation to mitochondria. In certain embodiments, the A44/K39 peptide portion comprises a functional fragment or variant of native A44 or K39 and has one or more of the following functions: capable of entering epithelial cells and, optionally, other cell types; capable of entering epithelial cells and, optionally other cell types by endocytosis; capable of translocating to mitochondria; capable of transitting at least one mitochondrial membrane; capable of transitting both mitochondrial membranes; capable of promoting or increasing expression of one or more anti-apoptotic gene (e.g., bcl-2, bcl-XL, NFkB, and mcl-1); capable of decreasing or promoting decrease in expression of one or more pro-apoptotic genes (e.g., bax, bak and TNFa); capable of increasing or promoting increase in expression of one or more pro-survival genes (e.g., rhoA, stat3, and cIAP1).

### III. Heterologous Agents

The disclosure provides conjugates comprising a heterologous agent portion. Without being bound by theory, the heterologous agent, when conjugated to the A44/K39 peptide portion can be carried into cells and localized to and/or into the mitochondria. The disclosure contemplates conjugates comprising any A44/K39 peptide portion associated with any heterologous agent (e.g., conjugates of the disclosure).

The disclosure provides conjugates for use for delivery into cells and tissues, particularly into cells and tissues and to (or into) mitochondria, such as across one or both mitochondrial membranes. Without being bound by theory, by providing heterologous agents complexed to an A44/K39 peptide portion, heterologous agent is delivered.

The heterologous agent portion may be a protein, peptide, polynucleotide, oligonucleotide, polymer or small organic or inorganic molecule. Generally, the heterologous agent is one with therapeutic or cell modulating activity that requires transport into cells to achieve. Below various categories of heterologous agent, as well as specific examples of heterologous agents are described. These specific examples of heterologous agents are merely illustrative. Conjugates of the disclosure comprising a A44/K39 peptide portion and a heterologous agent portion have substantial utility, for example, for delivering materials into cells and facilitating trafficking of materials to and, optionally into, mitochondria (e.g., across one or both mitochondrial membranes). It should be understood that the heterologous agent is heterologous to the A44/K39 peptide portion. In other words, the heterologous agent is not a functional portion of a gingipain protein (although, in certain embodiments, the conjugate may comprise such other portions of a gingipain protein). Moreover, in certain embodiments, the heterologous agent is not an agent that endogenously (in vivo under physiological conditions) binds to an A44 or K39 peptide.

A heterologous agent of the disclosure is an active agent. In other words, a heterologous agent has a biological activity other than as merely a short series of amino acid residues used solely to facilitate detection or purification, such as a myc or His tag (e.g., although it is appreciated that a conjugate may further include such a tag). In certain embodiments, a heterologous agent of the disclosure is not a fluorescent protein or a fluorescent dye. However, in certain embodiments, the conjugate further includes a fluorescent protein or a fluorescent dye (e.g., in addition to another heterologous agent). In certain embodiments, a heterologous agent is a therapeutic agent. In certain embodiments, a heterologous agent may be a pharmaceutical, a nutraceutical, or a therapeutically effective supplement or replacement of an endogenous component. In certain embodiments, the heterologous agent is an antioxidant.

Any type of heterologous agent may be used in accordance with the conjugates and methods described herein. In certain embodiments, the heterologous agent is a nucleic acid, a small molecule, a polymer, a polypeptide (such as an enzyme or antibody), or a peptide. In certain embodiments, following conjugation to a A44/K39 peptide portion, the heterologous agent retains at least 50%, 60%, 70%, 75%, 80%, or greater than 85% of a function, relative to the heterologous agent alone.

Proteins and Peptides

In certain embodiments, the heterologous agent portion is a protein or peptide. In the context of a conjugate of the disclosure, the protein or peptide maintains its functional activity, such as enzymatic activity, target binding and inhibitory activity, transcription factor activity, and the like. Exemplary categories of proteins and peptides that may serve as a heterologous agent are described in more detail below. In certain embodiments, the protein or peptide is one whose activity is needed in the mitochondria. For example, the protein or peptide may be one that, under naturally occurring circumstances, would be functional in mitochondria, and delivery is useful for augmenting or replacing activity that is supposed to be endogenously present in mitochondria. By way of further example, the protein or peptide may be one designed to inhibit activity of a target that is expressed or misexpressed in mitochondria, and delivery is useful for inhibiting that activity.

In certain embodiments, the heterologous agent is an enzyme or antibody.

a) Enzymes

In certain embodiments, the heterologous agent comprises an enzyme. Without being bound by theory, conjugates in which the heterologous agent is an enzyme are suitable for enzyme replacement strategies in which subjects are unable to produce an enzyme having proper activity (at all or, at least, in sufficient quantities) necessary for normal function and, in some case, essential for life.

When provided as a conjugate, the enzyme portion (heterologous agent comprising an enzyme) is delivered into cells and to mitochondria where it can provide needed enzymatic activity, such as at the mitochondria surface or within the mitochondria. In certain embodiments, the enzyme being delivered is one that is endogenously expressed in mitochondria of healthy subjects. Of course, it will be understood that the enzyme may, but need not be, endogenously expressed only in mitochondria. Moreover, throughout the application, it is understood that although the A44/K39 peptide portions (and conjugates of the disclosure) traffick to mitochondria, that does not mean that all of the administered conjugate localizes to mitochondria. Accordingly, such conjugates can deliver, optionally and in certain embodiments, heterologous agent to cytoplasm or to the surface of mitochondria.

An enzyme is a protein that can catalyze the rate of a chemical reaction within a cell. Enzymes are long, linear chains of amino acids that fold to produce a three-dimensional product having an active site containing catalytic amino acid residues. Substrate specificity is determined by the properties and spatial arrangement of the catalytic amino acid residues forming the active site. In certain embodiments, the enzyme does not comprise the catalytic domain of a gingipain protein.

As used herein an “enzyme” refers to a biologically active enzyme. The term “enzyme” further refers to “simple enzymes” which are composed wholly of protein, or “complex enzymes”, also referred to as “holoenzymes” which are composed of a protein component (the “apozyme”) and a relatively small organic molecule (the “co-enzyme”, when the organic molecule is non-covalently bound to the protein or “prosthetic group”, when the organic molecule is covalently bound to the protein).

As used herein the term an “enzyme” also refers to a gene for an enzyme and includes the full-length DNA sequence, a fragment thereof or a sequence capable of hybridizing thereto.

Classification of enzymes is conventionally based on the type of reaction catalyzed.

In certain embodiments, the heterologous agent comprises an enzyme. For example, the enzyme may be a human protein endogenously expressed in humans. Alternatively, the enzyme may be a non-human protein and/or a protein that is not endogenously expressed in humans.

The disclosure contemplates that sometimes a particular protein is not itself an enzyme, but is necessary for enzymatic or other catalytic or functional activity. Accordingly, in certain embodiments, the heterologous agent portion comprises a co-factor, accessory protein, or member of a multi-protein complex. Preferably, such a co-factor, accessory protein, or member of a multi-protein complex is a human protein or peptide. The protein or peptide should maintain its ability to bind to its endogenous cognate binding partners when provided as part of a conjugate of the disclosure.

b) Antibody

In another embodiment, a heterologous agent of the disclosure may be an antibody, such as, for example, an antibody that binds to a mitochondrial protein or an antibody that binds to and inhibits a protein misexpressed in mitochondria. The term “antibody” as used herein is intended to include antigen binding fragments thereof. Antibodies can be fragmented using conventional techniques and the fragments screened for utility in the same manner as is suitable for whole antibodies. For example, F(ab′)₂ fragments can be generated by treating antibody with pepsin. The resulting F(ab′)₂ fragment can be treated to reduce disulfide bridges to produce Fab′ fragments. Antibodies are further intended to include bispecific and chimeric molecules, as well as single chain (scFv) antibodies. Also included are trimeric antibodies, humanized antibodies, human antibodies, and single chain antibodies. All of these modified forms of antibodies as well as fragments of antibodies are intended to be included in the term “antibody”.

Small Molecules

In certain embodiments, the heterologous agent is a small organic or inorganic molecule. Such small molecules can be conjugated to the A44/K39 peptide portion, such as chemically conjugated. In certain embodiments, the small molecule is a small organic molecule. In certain embodiments, the small molecule is less than 1000, less than 750, less than 650, or less than 550 amu. In other embodiments, the small molecule is less than 500 amu, less than 400 amu, less than 300 amu. In certain embodiments, a heterologous agent of the disclosure may be a small organic molecule having multiple carbon-carbon bonds. Typically such compounds comprise one or more functional groups that mediate structural interactions with proteins, e.g., hydrogen bonding, and typically include at least an amine, carbonyl, hydroxyl or carboxyl group, and in some embodiments at least two of the functional chemical groups. The small molecule agents may comprise cyclic carbon or heterocyclic structures and/or aromatic or polyaromatic structures substituted with one or more chemical functional groups and/or heteroatoms.

In certain embodiments, the small molecule is a vitamin, such as vitamin E or vitamin C. In certain embodiments, the vitamin E is alpha-tocopherol. In certain embodiments, the small molecule is coenzyme Q10 (which may also be considered a cofactor).

In certain embodiments, the small molecule (or polypeptide or peptide) is an antibiotic.

In certain embodiments, the small molecule (or polypeptide or peptide) is an antioxidant. In other words, in certain embodiments, the heterologous agent is an antioxidant. Vitamin E is an example of such an antioxidant. Other exemplary antioxidants include glutathione, vitamin C, vitamin A, and vitamin E, as well as enzymes such as catalase, superoxide dismutase and various peroxidases.

Nucleic Acids

In certain embodiments, the heterologous agent comprises a polynucleotide, such as a mitochondrial DNA or an oligonucleotide, such as an antisense oligonucleotide.

In one embodiment, a heterologous agent of the disclosure may be a polynucleotide that encodes a polypeptide. In certain embodiments, the heterologous agent comprises a polynucleotide encoding a polypeptide, such as a polypeptide useful for decreasing oxidative stress and/or inhibiting apoptotic mechanisms. In certain embodiments, the heterologous agent comprises a polynucleotide encoding a polypeptide, such as a polypeptide useful for regulating the activity or expression of a protein present in mitochondria. In certain embodiments, the heterologous agent comprises a polynucleotide encoding a mitochondrial protein or polypeptide, such as a mitochondrial enzyme or subunit thereof.

In one embodiment, a heterologous agent of the disclosure may be an antisense oligonucleotide. By “antisense oligonucleotide,” it is meant a non-enzymatic nucleic acid compound that binds to a target nucleic acid by means of RNA-RNA, RNA-DNA or RNA-PNA (protein nucleic acid) interactions and alters the activity of the target nucleic acid (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can form a loop and binds to a substrate nucleic acid which forms a loop. Thus, an antisense molecule can be complementary to two (or more) non-contiguous substrate sequences, or two (or more) non-contiguous sequence portions of an antisense molecule can be complementary to a target sequence, or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151. Crooke, 2000, Methods Enzymol., 313, 3-45; Crooke, 1998, Biotech. Genet. Eng. Rev., 15, 121-157, Crooke, 1997, Ad. Pharmacol., 40, 1-49.

In other embodiments, a heterologous agent of the disclosure may be an siRNA, such as a short hairpin RNA (shRNA). The term “short interfering RNA,” “siRNA,” or “short interfering nucleic acid,” refers to any nucleic acid compound capable of mediating RNAi or gene silencing when processed appropriately be a cell. For example, the siRNA can be a double-stranded polynucleotide molecule comprising self-complementary sense and antisense regions, wherein the antisense region comprises complementarity to a target nucleic acid compound.

Production of the siRNAs can be carried out by chemical synthetic methods or by recombinant nucleic acid techniques. Endogenous RNA polymerase of the treated cell may mediate transcription in vivo, or cloned RNA polymerase can be used for transcription in vitro. As used herein, siRNA molecules need not be limited to those molecules containing only RNA, but may contain a DNA strand, several DNA nucleotides, and/or encompasses chemically-modified nucleotides and non-nucleotides. For example, siRNAs may include modifications to either the phosphate-sugar backbone or the nucleoside, e.g., to reduce susceptibility to cellular nucleases, improve bioavailability, improve formulation characteristics, and/or change other pharmacokinetic properties. To illustrate, the phosphodiester linkages of natural RNA may be modified to include at least one of a nitrogen or sulfur heteroatom. Modifications in RNA structure may be tailored to allow specific genetic inhibition while avoiding a general response to double stranded RNA (dsRNA). Likewise, bases may be modified to block the activity of adenosine deaminase. The dsRNAs may be produced enzymatically or by partial/total organic synthesis, any modified ribonucleotide can be introduced by in vitro enzymatic or organic synthesis. Methods of chemically modifying RNA molecules can be adapted for modifying dsRNAs (see, e.g., Heidenreich et al. (1997) Nucleic Acids Res. 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an siRNA can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration). In certain cases, the dsRNAs of the disclosure lack 2′-hydroxy(2′-OH) containing nucleotides.

In some embodiments, the antisense oligonucleotide is a morpholino molecule that sterically blocks the binding of a protein or nucleic acid to a target RNA or DNA sequence. In some embodiments, the morpholino also triggers degradation of the target RNA or DNA sequence. In some embodiments, the morpholino molecule comprises 20-30 nucleotides. In other embodiments, the morpholino molecule comprises 23-27 nucleotides. In other embodiments, the morpholino molecule comprises 25 nucleotides.

In some embodiments, the antisense oligonucleotides of the present disclosure include a nucleotide analog having a constrained furanose ring conformation, such as Locked Nucleic Acids (LNAs). In LNAs, a 2′-hydroxyl group is linked to the 3′ or 4′ carbon atom of the sugar ring thereby forming a bicyclic sugar moiety.

In some embodiments, in modified oligonucleotide, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA nucleotides include, but are not limited to, U.S. Pat. Nos. 5,539,082; 5,714,331; and 5,719,262, each of which are herein incorporated by reference. Further teaching of PNA nucleotides can be found in Nielsen et al., Science, 1991, 254, 1497-1500.

Polymers

In certain embodiments, the heterologous agent is a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 daltons (or about 1,000 to about 15,000, and 2,000 to about 12,500) can be used.

In certain embodiments, the heterologous agent is a water soluble polymer, e.g., a hydrophilic polyvinyl polymer, e.g. polyvinylalcohol or polyvinylpyrrolidone. In certain embodiments, the heterologous agent is polyethylene glycol (PEG), polypropylene glycol, or polyoxyethylenated polyol. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon. In certain embodiments, the heterologous agent is polyethylene glycol (PEG). Heterologous agents include vitamins, such as vitamin E, A or C which may also be examples of antioxidants. A heterologous agent of the disclosure may be a therapeutic or therapeutically useful for a mitochondrial disease. One therapeutic category of mitochondrial diseases is associated with cellular degeneration, which is often mediated by oxidative stress and apoptotic mechanisms. Cardiovascular diseases like atherosclerosis, ischemia/reperfusion injury, heart failure, stroke, and traumatic brain injury; neurodegenerative diseases like Alzheimer's disease, Parkinson's disease, Huntington's disease, amyotrophic lateral sclerosis (ALS), autism, and muscular dystrophy; chronic autoimmune inflammatory diseases like rheumatoid arthritis; metabolic diseases like diabetes and obesity; and aging could all be considered in this category. Another therapeutic category of mitochondria diseases is associated with hyperproliferative states, e.g., cancers.

In certain embodiments, the heterologous agent is useful for decreasing oxidative stress and/or inhibiting apoptotic mechanisms. In certain embodiments, the heterologous agent is useful for replacing an inactive or underactive protein (or subunit thereof) normally functional in mitochondria. In certain embodiments, the heterologous agent targets a protein (or subunit thereof) present in mitochondria, such as to inhibit or down-regulate its activity. In certain embodiments, the heterologous agent targets a protein (or subunit thereof) present in mitochondria, such as to enhance or up-regulate its activity.

In certain embodiments, the heterologous agent is an antioxidant.

Examples of therapeutics for a mitochondrial disease that may be used as heterologous agents of the disclosure include, but are not limited to, vitamin E, vitamin C, cytochrome C oxidase, glutathione, vitamin A, catalase, superoxide dismutase, peroxidase, coenzyme Q10, L-Carnitine, adenylate kinase isozyme 2 (Dak2), and EPI-743. Additional agents for use in treating mitochondrial diseases are known to one of skill in the art. See. e.g., U.S. Patent Application Publication No. 20100210569, herein incorporated by reference in its entirety.

### IV. Conjugates

The disclosure provides conjugates composed of at least two components: an A44/K39 peptide portion component (e.g., a polypeptide comprising or consisting of an A44 or A39 peptide, or a functional fragment thereof, or a functional variant thereof) and a heterologous agent component. Conjugates of the disclosure are compounds comprising these two components associated therewith, such as covalently interconnected. In certain embodiments, the components are covalently attached as fusions (directly or indirectly, such as via a linker) via a peptide bond or are interconnected via chemical methods (directly or indirectly, such as via a linker). The term “conjugate(s) of the disclosure” is used herein to refer to any of the conjugates described herein comprising an A44/K39 peptide portion associated with, such as covalently associated with, at least one heterologous agent, and includes all combinations of peptide sequences, heterogogous agents, further modifications, and linkers. In other words, conjugates of the disclosure refer to a compound comprising an A44/K39 peptide portion and a heterologous agent portion, wherein the portions are associated, such as covalently interconnected.

Any of the A44/K39 peptide portions may be combined with any of the heterologous agent portions. Suitable conjugates retain one or more functions of the A44/K39 peptide portions and one or more functions of the heterologous agent portion. Conjugates may be associated directly or via a linker. Conjugates may be PEGylated or otherwise further modified. Conjugates may be provided as isolated or purified conjugates and/or as a composition comprising a conjugate.

As disclosed herein, exemplary A44 or K39 peptides (e.g., exemplary A44/K39 peptide portions) capable of mediating cellular uptake, such as into epithelial cells, and translocation to mitochondria include peptides comprising the amino acid sequence of SEQ ID NO.: 1, SEQ ID NO.: 2, SEQ ID NO.: 3 or SEQ ID NO.: 4 are provided. Accordingly, conjugates comprising such a peptide, or functional fragments thereof or functional variants thereof, are particularly useful for, but not limited to, delivering a heterologous agent inside a cell and, in particular, to mitochondria. Such conjugates are suitable for a variety of in vitro or in vivo applications, such as in the contexts of mitochondrial diseases and disorders, and in cells with mitochondrial dysfunctional or deficiency. Such conjugates are specifically contemplated.

As disclosed herein, exemplary fragments of A44 or K39 peptides (e.g., A44/K39 peptide portions) capable of mediating cellular uptake, such as into epithelial cells, including peptides comprising the amino acid sequence of SEQ ID NO.: 3, SEQ ID NO.: 4, SEQ ID NO.: 7, SEQ ID NO.: 8, SEQ ID NO.: 9 and SEQ ID NO.: 10 are provided. Accordingly, conjugates comprising such peptides, or functional fragments or variants thereof, are particularly useful for, but not limited to, delivering a heterologous agent inside a cell, where the agent exerts its biological activity, e.g. in the cytoplasm of a cell. Such conjugates are applicable for use in a variety of cells and disease states as a general purpose drug delivery vehicle.

As disclosed herein, exemplary fragments of A44 or K39 peptides (e.g., A44/K39 peptide portions) capable of translocating to mitochondria, including peptides comprising the amino acid sequence of SEQ ID NO.: 3, SEQ ID NO.: 4, SEQ ID NO.: 11, SEQ ID NO.: 12, SEQ ID NO.: 13 and SEQ ID NO.: 14, as well as peptides comprising the amino acid sequence (PN)₅P or (PN)₄P, are provided. Accordingly, conjugates comprising such a peptide are particularly useful for, but not limited to, delivering a heterologous agent to mitochondria, where it can exert it biological activity. Such conjugates used in combination with liposome-based vesicles for example, or another cell-permeable drug delivery technology, are useful in a variety of diseases, in particular mitochondrial diseases and disorders and in cells with mitochondrial dysfunctional or deficiency. Alternatively, nucleic acid-based approaches, using e.g., DNA or RNA viral vectors containing a recombinant nucleic acid encoding such a conjugate can be employed, such that upon viral entry the conjugate is expressed using host machinery. Such conjugates could be applicable for use in a variety of cells and disease states, in particular mitochondrial diseases and disorders.

A44/K39 peptide portions suitable for use in conjugates of the disclosure have one or more functional features of full length, native A44 or K39. For example, in certain embodiments, suitable A44/K39 peptide portions and/or conjugates have one or more of the following functions: capable of promoting entry into cells, such as epithelial cells; capable of promoting translocation to mitochondria; capable of transiting one or both mitochondrial membranes; capable of mediating anti-apoptotic effects, by e.g. inducing a change in gene expression in one or more genes; capable of promoting expression or upregulation of one or more pro-survival genes; capable of inhibiting expression or downregulating one or apoptotic genes.

In certain embodiments, conjugates comprising such an A44/K39 peptide portion induce a gene expression change in genomic DNA and/or mitochondrial DNA.

Moreover, the association between the two components may persist following internalization into a cell or may be transient. For example, if the components are covalently linked via a linker, the linker may be a cleavable linker, such that the association may be disrupted after successful deliver of the heterologous agent into a cell or into mitochondria (e.g., once inside the cell, the complex may optionally be disrupted). Alternatively, the association may be non-cleavable such that the two portions of the conjugate remain associated.

Conjugates of the disclosure can be modified, e.g., with a moiety that improves its stabilization and/or retention in circulation, e.g., in blood, serum, lymph, bronchoalveolar lavage, or other tissues, e.g., by at least 1.5, 2, 5, 10, or 50 fold.

For example, a conjugate of the disclosure can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 daltons (or about 1,000 to about 15,000, and 2,000 to about 12,500) can be used. Similarly, in certain embodiments, such polymers may be the heterologous agent portion of the conjugate.

For example, a conjugate of the disclosure can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

In certain embodiments, the polymer is polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161).

In certain embodiments, the conjugate further comprises a moiety to increase detection, uptake/administration, production, or purification, e.g., polypeptides that include epitope tags, such as HA and myc tags, as well as the Fe region of an immunoglobulin or all or a portion of HSA. In certain embodiments, the conjugate further comprises an Fe portion of an antibody. In certain embodiments, the conjugate further comprises a fluorescent label, such as a fluorescent dye or fluorescent bead. The disclosure contemplates conjugates in which the A44/K39 peptide portion and the heterologous agent portion are associated by a covalent or non-covalent linkage. In either case, the association may be direct or via one or more additional intervening linkers or moieties.

In some embodiments, an A44/K39 peptide portion and a heterologous agent portion are associated through chemical or proteinaceous linkers or spacers. Exemplary linkers and spacers include, but are not restricted to, substituted or unsubstituted alkyl chains, polyethylene glycol derivatives, amino acid spacers, sugars, or aliphatic or aromatic spacers common in the art.

Suitable linkers include, for example, homobifunctional and heterobifunctional cross-linking molecules. The homobifunctional molecules have at least two reactive functional groups, which are the same. The reactive functional groups on a homobifunctional molecule include, for example, aldehyde groups and active ester groups. Homobifunctional molecules having aldehyde groups include, for example, glutaraldehyde and subaraldehyde.

Homobifunctional linker molecules having at least two active ester units include esters of dicarboxylic acids and N-hydroxysuccinimide. Some examples of such N-succinimidyl esters include disuccinimidyl suberate and dithio-bis-(succinimidyl propionate), and their soluble bis-sulfonic acid and bis-sulfonate salts such as their sodium and potassium salts.

Heterobifunctional linker molecules have at least two different reactive groups. Examples of heterobifunctional reagents containing reactive disulfide bonds include N-succinimidyl 3-(2-pyridyl-dithio)propionate (Carlsson et al., 1978. Biochem. J., 173:723-737), sodium S-4-succinimidyloxycarbonyl-alpha-methylbenzylthiosulfate, and 4-succinimidyloxycarbonyl-alpha-methyl-(2-pyridyldithio)toluene. Examples of heterobifunctional reagents comprising reactive groups having a double bond that reacts with a thiol group include succinimidyl 4-(N-maleimidomethyl)cyclohexahe-1-carboxylate and succinimidyl m-maleimidobenzoate. Other heterobifunctional molecules include succinimidyl 3-(maleimido)propionate, sulfosuccinimidyl 4-(p-maleimido-phenyl)butyrate, sulfosuccinimidyl 4-(N-maleimidomethyl-cyclohexane)-1-carboxylate, maleimidobenzoyl-5N-hydroxy-succinimide ester.

Other means of cross-linking proteins utilize affinity molecule binding pairs, which selectively interact with acceptor groups. One entity of the binding pair can be fused or otherwise linked to the A44/K39 peptideportion and the other entity of the binding pair can be fused or otherwise linked to the heterologous agent portion. Exemplary affinity molecule binding pairs include biotin and streptavidin, and derivatives thereof; metal binding molecules; and fragments and combinations of these molecules. Exemplary affinity binding pairs include StreptTag (WSHPQFEK)/SBP (streptavidin binding protein), cellulose binding domain/cellulose, chitin binding domain/chitin, S-peptide/S-fragment of RNAseA, calmodulin binding peptide/calmodulin, and maltose binding protein/amylose.

In one embodiment, the A44/K39 peptide portion and the heterologous agent portion are linked by ubiquitin (and ubiquitin-like) conjugation. In other embodiments, the A44/K39 peptide portion and the heterologous agent portion may be fused through an enzymatic reaction, through a disulfide bond, or through an artificial amino acid.

All interconnections may be, in certain embodiments, via a linker. Accordingly, conjugates of the disclosure may comprise a linker. Moreover, the orientation (e.g., which portion is N-terminal versus C-terminal relative to the other portion) may vary. In fact, a heterologous agent may be interconnected (directly or indirectly) to the A44/K39 peptide portion via an internal amino acid of the A44/K39 peptide portion, or may be interconnect (directly or indirectly) to the N- or C-terminal amino acid.

In another embodiment, the A44/K39 peptide portion and heterologous agent portion are interconnected via a peptide bond, such as made as a fusion protein or otherwise interconnected recombinantly.

The disclosure also provides nucleic acids encoding an A44/K39 peptide portion and a heterologous agent portion. The complex (e.g., the conjugate) of an A44/K39 peptide portion and heterologous agent portion can be expressed as a fusion protein, optionally separated by a peptide linker. The peptide linker can be cleavable or not cleavable. A nucleic acid encoding a fusion protein can express the fusion in any orientation. For example, the nucleic acid can express an N-terminal A44/K39 peptide portion fused to a C-terminal heterologous agent portion, or can express an N-terminal heterologous agent portion fused to a C-terminal A44/K39 peptide portion.

A nucleic acid encoding an A44/K39 peptide portion can be on a vector that is separate from a vector that carries a nucleic acid encoding a heterologous agent portion. The A44/K39 peptide portion and the heterologous portion can be expressed separately, and complexed (including chemically linked) prior to introduction to a cell for delivery. The isolated conjugate can be formulated for administration to a subject, as a pharmaceutical composition.

The disclosure also provides host cells comprising a nucleic acid encoding the A44/K39 peptide portion or the heterologous agent portion, or comprising the complex as a fusion protein. The host cells can be, for example, prokaryotic cells (e.g., E. coli) or eukaryotic cells. The two portions can be made in the same or in different host cells. In some embodiments, only the A44/K39 peptide portion is a polypeptide, and the heterologous agent is made by suitable methods and later associated with the A44/K39 peptide portion.

In certain embodiments, the recombinant nucleic acids encoding a conuugate, or the portions thereof, may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used. In certain aspects, this disclosure relates to an expression vector comprising a nucleotide sequence encoding a conjugate of the disclosure polypeptide and operably linked to at least one regulatory sequence. Regulatory sequences are art-recognized and are selected to direct expression of the encoded polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

The disclosure also provides host cells comprising or transfected with a nucleic acid encoding the conjugate as a fusion protein. The host cells can be, for example, prokaryotic cells (e.g., E. coli) or eukaryotic cells. Other suitable host cells are known to those skilled in the art.

In addition to the nucleic acid sequence encoding the conjugate or portions of the conjugate, a recombinant expression vector may carry additional nucleic acid sequences, such as sequences that regulate replication of the vector in a host cells (e.g., origins of replication) and selectable marker genes. The selectable marker gene facilitates selection of host cells into which the vector has been introduced. Exemplary selectable marker genes include the ampicillin and the kanamycin resistance genes for use in E. coli.

The present disclosure further pertains to methods of producing fusion proteins of the disclosure. For example, a host cell transfected with an expression vector can be cultured under appropriate conditions to allow expression of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art. The polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptides. In a preferred embodiment, the polypeptide is a fusion protein containing a domain which facilitates its purification.

A nucleic acid encoding an A44/K39 peptide portion can be on a vector that is separate from a vector that carries a nucleic acid encoding a heterologous agent portion. The portions of the conjuate can be expressed separately and complexed prior to introduction to a cell for delivery. The isolated conjugate can be formulated for administration, as a pharmaceutical composition. As noted above, when expressed separately, the A44/K39 peptide portion and the heterologous agent portion may be expressed using the same or differing vectors and/or using the same or differing host cells.

Recombinant nucleic acids of the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of cukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in cukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

It should be understood that fusion polypeptides or protein of the present disclosure can be made in numerous ways.

In certain embodiments, a conjugate of the disclosure is formed under conditions where the linkage (e.g., by a covalent or non-covalent linkage) is formed, while the activity of the heterologous agent portion is maintained. In certain embodiments, the conjugate maintains at least 50% of a native activity (e.g., of at least one native activity) of the heterologous agent portion alone. For example, where the heterologous agent portion is an enzyme, the conjugate retains at least 50% of the native activity of that enzyme.

### V. Methods of Production

The disclosure provides nucleic acid constructs comprising a nucleotide sequence that encodes the (i) conjugate described herein or (ii) the peptide portion of the conjugate described herein, wherein the nucleic acid construct encodes a conjugate capable of entering a cell by endocytosis and localizing to mitochondria. The disclosure also provides host cells comprising the nucleic acid described herein.

The disclosure provides methods of producing the conjugates described herein. In certain embodiments, the conjugate is made recombinantly in a host cell using standard techniques. In certain embodiments, the method of producing the conjugates comprises culturing the host cell described herein under conditions for producing said conjugate. In certain embodiments, the method further comprises isolating said conjugate, such that the conjugates are substantially purified or pyrogen free.

In addition, the conjugates described herein can also be chemically synthesized using techniques known in the art such as conventional Merrifield solid phase f-Moc or t-Boc chemistry.

In certain embodiments, where the A44/K39 peptide portion and the heterologous agent portion are both polypeptides, the conjugate can be made as an in-frame fusion, such as where both portions are encoded by nucleic acid present in the same vector, in the presence or absence of nucleic acid encoding a linker, such as a glycine/serine linker. Alternatively, the two portions can be made recombinantly from separate vectors and interconnected via chemical conjugation. In other embodiments, the A44/K39 peptide portion is amplified from a sample containing full length gingipain protein using, for example, PCR, and the PCT amplified A44/K39 peptide portion can then be interconnected to a heterologous agent portion using recombinant or chemical means in the presence or absence of a linker.

In embodiments in which the heterologous agent is a small molecule or nucleic acid, the heterologous agent is made or obtained using standard methods and the A44/K39 peptide portion can be made using, for example, any of the foregoing methods. Once made, the two portions can be interconnected directly or via a linker using, for example, an appropriate chemical conjugation approach. Non-covalent association is also contemplated.

### VI. Methods of Use

The disclosure provides a variety of in vitro and in vivo methods of use. For example, conjugates of the disclosure can be used in vitro as reagents for studying mitochondrial function or dysfunction. By way of example, a conjugate of the disclosure is suitable for delivering a heterologous agent to mitochondria in cells in vitro. Such cells may be models of mitochondrial dysfunction or may be healthy cells, and administering (e.g., contacting) the cells with a conjugate including a particular heterologous agent is useful for evaluating the impact of the heterologous agent and delivery of that agent to mitochondria in improving one or more parameters of the cell. In one example, the heterologous agent is an agent that is already a candidate for treatment of a mitochondrial dysfunction or is a known neutraceutical, and the in vitro methods are useful for evaluating the improvement in activity when the agent is directed to mitochondria using the A44/K39 peptide portions provided herein. In such an example, activity can be assessed relative to administering the heterologous agent alone (e.g., not in the presence of a conjugate of the disclosure).

In other embodiments of research uses for conjugates of the disclosure, the conjugate is used to identify native binding partners of a particular agent in mitochondria or a native binding partner of a particular A44/K39 peptide portion (e.g., a particular fragment of native A44 or K39). In other words, a conjugate of the disclosure is useful for identifying binding partners specifically in mitochondria. Similarly, evaluation of binding interactions can be performed in cells, such as healthy epithelial cells or stable, non-disease cell lines, or in cells that are models of mitochondrial dysfunction (e.g., cells from a patient or from an animal model of mitochondrial dysfunction or a cell-based model of mitochondrial dysfunction). Evaluation may be performed in both healthy cells and in models of dysfunction to identify differences in interactions that may be indicative of the mechanism of action in the disease state.

In certain embodiments, the foregoing methods are applied to epithelial cells in culture. In certain embodiments, the foregoing methods are applied to cells having mitochondrial dysfunction.

Other in vitro and in vivo methods are described herein. It is contemplates that many of the methods of the disclosure may be performed in vitro or in vivo. For example, methods of delivering a heterologous agent (e.g., present in the context of a conjugate) into a cell (e.g., an epithelial cell) and/or to or into mitochondria can be performed in vitro or in vivo (administered to the subject. In certain embodiments, a subject is a subject in need thereof (e.g., a subject having a mitochondrial disease, condition or dysfunction; a subject having cells characterized by mitochondrial dysfunction).

Conjugates of the disclosure have, in certain embodiments, one or more functional characteristics of the A44/K39 peptide portion and one or more functional characteristics of the heterologous agent. For example, in certain embodiments, a conjugate of the disclosure has any one or more of the following functional properties of a A44/K39 peptide portion: ability to enter epithelial cells and, optionally, other cell types; ability to enter epithelial cells and, optionally other cell types by endocytosis; ability to traffic to the mitochondria; ability to transit at least one mitochondrial membrane; ability to transit both mitochondrial membrane; the ability to promote increase in expression of one or more anti-apoptotic gene (e.g., bcl-2, bcl-XL, NFkB, and mcl-1), relative to an untreated cell that is not contacted with the A44/K39 peptide portion of the conjugate and/or with the conjugate; the ability to promote decrease in expression of one or more pro-apoptotic genes (e.g., bax, bak and TNFa) relative to an untreated cell that is not contacted with the A44/K39 peptide portion of the conjugate and/or with the conjugate; the ability to promote increase in expression of one or more pro-survival genes (e.g., rhoA, stat3, and cIAP1) relative to an untreated cell that is not contacted with the A44/K39 peptide portion of the conjugate and/or with the conjugate.

Conjugates of the disclosure may comprise any one or more of the foregoing features and may be described using any combination of structural and/or functional features. In certain embodiments, conjugates of the disclosure are capable of entering cells, such as epithelial cells, such as by endocytosis, and trafficking to the mitochondria. Conjugates of the disclosure can be used in any of the methods described herein.

In certain embodiments, the disclosure provides methods of targeting a conjugate and/or heterologous agent to the mitochondria. In certain embodiments, a conjugate alters mitochondrial function. The methods may be performed in vitro or in vivo. In certain embodiments of the methods, administering the conjugate results in altered gene expression or protein activity of a mitochondrial protein. In certain embodiments of the methods, administering the conjugate results in increased expression of one or more anti-apoptotic genes, e.g., bcl-2, bcl-XL. NFkB, and mcl-1, relative to an untreated cell. In certain embodiments of the methods, administering the conjugate results in decreased expression of one or more pro-apoptotic genes, e.g., bax, bak and TNFa, relative to an untreated cell. In certain embodiments of the methods, administering the conjugate results in increased expression of one or more pro-survival genes, e.g., rhoA, stat3, and cIAP1, relative to an untreated cell.

The disclosure provides a variety of cell types applicable for the methods disclosed herein. In certain embodiments, the cell is an endodermal-derived cell, such as a pancreatic cell, a lung cell, or a thyroid cell. In certain embodiments, the cell is a mesodermal-derived cell, such as a fibroblast, an endothelial cell, a myocyte, hepatocyte, an adipocyte or a T cell. In certain embodiments, the cell is an ectodermal-derived cell, such as an epithelial cell, a neuronal cell or a glial cell. In certain embodiments, the cell is an epithelial cell. In another preferred embodiment, the cell is a human cell. In certain embodiments of the methods, the cell is characterized by a mitochondrial dysfunction and/or is a cell from or of a subject having a mitochondrial disorder or condition.

The disclosure provides methods of treating a subject in need of treatment for a mitochondrial disease or condition. In certain related embodiments of the methods, the disease or condition is characterized by increased oxidative damage in a cell and/or increased cell death, for example a disease or condition characterized by degeneration e.g., cardiovascular diseases such as atherosclerosis, ischemia/reperfusion injury, heart failure, stroke, and traumatic brain injury: neurodegenerative diseases such as Alzheimer's disease, Parkinson's disease, Huntington's disease, amyotrophic lateral sclerosis (ALS), autism, and muscular dystrophy; chronic autoimmune inflammatory diseases such as rheumatoid arthritis; and metabolic diseases such as diabetes and obesity. In certain embodiments of the methods, wherein the disease or condition is characterized by increased cell death, the conjugate comprises a heterologous agent that promotes cell survival.

In certain embodiments of the methods, the disclosure provides for administering a conjugate comprising a heterologous agent such as the antioxidant vitamin E to reduce accumulation of reactive oxygen species. Given that numerous mitochondrial conditions are caused or exacerbated by increased oxidative damage, delivery of an antioxidant, such as vitamin E, to mitochondria is a suitable therapeutic. Accordingly, the disclosure provides methods of treating a mitochondrial disease or condition by administering a conjugate in which the heterologous agent is an antioxidant, such as vitamin E. Similarly, the disclosure provides methods of reducing accumulation of reactive oxygen species, comprising administering a conjugate of the disclosure, wherein the heterologous agent portion comprises an antioxidant, such as vitamin E.

In certain embodiments of the methods, the disease or condition is characterized by a hyperproliferative state, e.g., cancer. In certain embodiments of the methods, wherein the disease or condition is characterized by hyperproliferative, the conjugate comprises an A44/K39 peptide portion that is not capable of inducing expression of anti-apoptotic genes. In certain embodiments of the methods, wherein the disease or condition is characterized by hyperproliferative, the conjugate comprises a heterologous agent that promotes cell death. In some embodiments, the agents of the present disclosure may be used to treat several forms of cancer. These cancers include, but are not limited to: prostate cancer, bladder cancer, lung cancer (including small cell and non-small cell), colon cancer, kidney cancer, liver cancer, breast cancer, cervical cancer, endometrial or other uterine cancer, ovarian cancer, testicular cancer, cancer of the penis, cancer of the vagina, cancer of the urethra, gall bladder cancer, esophageal cancer, or pancreatic cancer. Additional cancer types include cancer of skeletal or smooth muscle, stomach cancer, cancer of the small intestine, cancer of the salivary gland, anal cancer, rectal cancer, thyroid cancer, parathyroid cancer, pituitary cancer, and nasopharyngeal cancer.

“Treating” or “treatment” or “alleviation” refers to both therapeutic treatment and prophylactic or preventative measures, wherein the object is to slow down (lessen) the progression of a disease. “Diagnosing” refers to the process of identifying or determining the distinguishing characteristics of a disease. For cancer therapy, the process of diagnosing is sometimes also expressed as staging or tumor classification based on severity or disease progression.

For example, a subject or mammal is successfully “treated” if, according to the method of the present disclosure, after receiving a therapeutic amount of an agent, the patient shows observable and/or measurable reduction in or absence of one or more of the symptoms of the disease. For example, these may include for cancer therapy, these may include the following: reduction in the number of tumor cells or absence of such cells; reduction in the tumor size; inhibition (i.e., slow to some extent and preferably stop) of tumor cell infiltration into peripheral organs including the spread of cancer into soft tissue and bone; inhibition (i.e., slow to some extent and preferably stop) of tumor metastasis; inhibition, to some extent, of tumor growth; and/or relief to some extent, of one or more of the symptoms associated with the specific cancer; reduced morbidity and mortality, and improvement in quality of life issues. To the extent such agent may prevent growth and/or kill existing cancer cells, it may be cytostatic and/or cytotoxic. Reduction of these signs or symptoms may also be felt by the patient.

In certain embodiments, the disclosure provides a method for treating a mitochondrial disorder or mitochondrial dysfunction in a cell, the method comprising administering an effective amount of a conjugate of the disclosure. The disclosure also provides a method for delivering a heterologous agent to mitochondria in a cell, such as to deliver a heterologous agent across one or both mitochondrial membranes. The disclosure provides a method of preventing cell death or promoting cell survival in a cell. The disclosure provides a method of preventing oxidative stress in a cell or for reducing reactive oxygen species. The disclosure provides a method of reducing the number of mitochondria undergoing mitochondrial permeability transitioning (MPT) or preventing mitochondrial permeability transitioning in a cell. The disclosure provides a method for delivering an agent into a cell and to mitochondria in a cell. The disclosure provides for a method of preventing or decreasing oxidative stress in a cell. In certain embodiments, a conjugate of the disclosure could be used in a protein-replacement therapy, such as to provide an enzyme that is inactive or has decreased function in a cell.

In certain embodiments, a conjugate of the disclosure can be used for decreasing mitochondrial dysfunction by minimizing mitochondrial Ca2+ overload, decreasing mitochondrial ROS accumulation, or improving mitochondrial energy production. See, U.S. Pat. No. 8,470,861, herein incorporated by reference in its entirety. These methods may be used with conjugates comprising heterologous agents such as vitamin E, vitamin C, cytochrome C oxidase, coenzyme Q10, L-carnitine, or EPI-743.

Generally, a heterologous agent that promotes cell survival may be used to treat degenerative disorders, while a heterologous agent that promotes death may be used to treat hyperproliferative disorders. In certain embodiments, a conjugate of the disclosure comprising a heterologous agent such as vitamin E, vitamin C, cytochrome C oxidase, coenzyme Q10, L-carnitine, EPI-743, or a pro-survival protein, e.g., rhoA, stat3, and cIAP1, is used to treat a degenerative disease, such as Huntington's disease. In certain embodiments, a conjugate of the disclosure comprising AK2 is used to treat reticular dysgenesis (RD). In certain embodiments, a conjugate of the disclosure comprising a heterologous agent encoding a growth factor, is used to treat a degenerative disease. In certain embodiments, a conjugate of the disclosure comprising a heterologous agent encoding glial cell line derived neutrotrophic factor (GDNF), is used to treat a degenerative disease, such as Parkinson's disease. In certain embodiments, a conjugate of the disclosure comprising a heterologous agent encoding vascular endothelial growth factor (VEGF), is used to treat a degenerative disease, such as Amyotrophic lateral sclerosis (ALS). In certain embodiments, a conjugate of the disclosure comprising a pro-apoptotic protein, such as bax, bak or TNFa, is used to treat a hyperproliferative disorder such as cancer.

The disclosure also provides screening or predictive methods. The disclosure provides for a method of predicting an effect of the conjugate of the disclosure on a cell of a subject in vivo, the method comprising i) obtaining a cell from a subject; ii) exposing the cell obtained from the subject to the conjugate; and iii) assaying for a pharmacological or toxicological effect of the conjugate on the cell relative to a control cell not exposed to the conjugate (or exposted to a heterologous agent provided in the absence of a A44/K39 peptide portion). In certain embodiments of the methods, the pharmacological effect is a change in mitochondrial activity or mitochondrial gene expression.

The disclosure provides for a method of identifying an effect of a conjugate disclosed herein, the method comprising exposing a cell to the conjugate and assaying for a pharmacological or toxicological effect of the conjugate on the cell relative to a control cell not exposed to the conjugate. In certain embodiments of the methods, the pharmacological effect is a change in mitochondrial activity or mitochondrial gene expression. Thus, the conjugates of the disclosure can be used in a screening assay.

Assessment of the activity of the candidate conjugate generally involves combining cells with the candidate, detecting and/or measuring any change in the morphology, marker phenotype, or metabolic activity of the cells that is attributable to the candidate (compared with untreated cells or cells treated with an inert compound or cells treated with heterologous agent is the absence of a A44/K39 portion), and then correlating the effect of the candidate agent with the observed change. The reader is referred generally to the standard textbook In Vitro Methods in Pharmaceutical Research, Academic Press, 1997, and U.S. Pat. No. 5,030,015. In some embodiments, the conjugates of the disclosure evaluated for potential cytotoxicity (Castell et al., In: In Vitro Methods in Pharmaceutical Research, Academic Press, 375-410, 1997; and Cell Encapsulation Technology and Therapeutics, Kuhtreiber et al. eds., Birkhauser, Boston, Mass., 1999), which are herein incorporated by reference in their entirety. Cytotoxicity can be determined in the first instance by detecting and/or measuring the effect on cell viability, morphology, leakage of enzymes into the culture medium, and/or induction of apoptosis (indicated by cell rounding, condensation of chromatin, and nuclear fragmentation). In certain embodiments, cytotoxicity may be assessed by observation of vital staining techniques, ELISA assays, immunohistochemistry, and the like or by analyzing the cellular content of the culture, e.g., by total cell counts, and differential cell counts or by metabolic markers such as MTT and XTT.

In certain embodiments, conjugates of the disclosure are suitable for evaluating the improvement in activity of a heterologous agent delivered to mitochondria versus that activity of the heterologous agent alone.

### VII. Methods of Administration

Various delivery systems are known and can be used to administer the conjugates of the disclosure, whether in vitro, ex vivo or in vivo. Any such methods may be used to administer any of the conjugates described herein. Conjugates of the disclosure include conjugates comprising any of the adhesion A44/K39 peptide portions and any of the heterologous agent portions. Moreover, any of the methods of administration described herein can be used to deliver conjugates of the disclosure, including conjugates that are isolated or purified. Moreover, any of the methods of administration described herein can be used to deliver conjugates of the disclosure that are formulated in a pharmaceutically acceptable carrier (e.g., compositions or pharmaceutical compositions).

Methods of introduction can be enteral or parenteral, including but not limited to, intradermal, intramuscular, intraperitoneal, intramyocardial, intravenous, subcutaneous, pulmonary, intranasal, intraocular, epidural, and oral routes. The conjugates may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local.

In certain embodiments, the conjugate is administered intravenously. In certain embodiments, the conjugate is administered subcutaneously.

In certain embodiments, it may be desirable to administer the conjugates of the disclosure locally to the area in need of treatment; this may be achieved, for example, and not by way of limitation, by local infusion during surgery, by means of a catheter, or by means of an implant, the implant being of a porous, non-porous, or gelatinous material, including membranes, such as sialastic membranes, fibers, or commercial skin substitutes.

Note that the disclosure contemplates methods in which conjugates are administered, at the same or different times, via one than one route of administration. For example, the disclosure contemplates a regimen in which conjugates are administered systemically, such as by intravenous infusion, in combination with local administration.

In other embodiments, the conjugates of the disclosure can be delivered in a vesicle, in particular, a liposome (see Langer, 1990, Science 249:1527-1533). In yet another embodiment, the conjugates of the disclosure can be delivered in a controlled release system. In another embodiment, a pump may be used (see Langer, 1990, supra). In another embodiment, polymeric materials can be used (see Howard et al., 1989, J. Neurosurg. 71:105). In certain specific embodiments, the conjugates of the disclosure can be delivered intravenously.

In certain embodiments, the conjugates are administered by intravenous infusion. In certain embodiments, the conjugates are infused over a period of at least 10, at least 15, at least 20, or at least 30 minutes. In other embodiments, the conjugates are infused over a period of at least 60, 90, or 120 minutes.

In certain embodiments, conjugates of the disclosure are used in vitro or ex vivo, such as to modify cells ex vivo. In certain embodiments, conjugates are administered to such cells by contacting cells in culture with a conjugate of the disclosure (e.g., by adding conjugate to cell culture media).

The foregoing applies to any of the conjugates, compositions, and methods described herein. The disclosure specifically contemplates any combination of the features of such conjugates, compositions, and methods (alone or in combination) with the features described for the various pharmaceutical compositions and route of administration described in this section.

### VIII. Pharmaceutical Compositions

In certain embodiments, the subject conjugates of the present disclosure are formulated with a pharmaceutically acceptable carrier (e.g., formulated with one or more pharmaceutically acceptable carriers and/or excipients). In other words, in certain embodiments, the disclosure provides compositions comprising a conjugate of the disclosure (including an isolated or purified conjugate) formulated with one or more pharmaceutically acceptable carriers and/or excipients. One or more conjugates can be administered alone or as a component of a pharmaceutical formulation (composition). Any of the conjugates described herein may be formulated, as described herein. The conjugates may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject conjugates include, for example, those suitable for oral, nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, methods of preparing these formulations or compositions include combining another type of therapeutic agents and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject conjugate therapeutic agent as an active ingredient. Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In solid dosage forms for oral administration (capsules, tablets, pills, dragees, powders, granules, and the like), one or more conjugate therapeutic agents of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

In certain embodiments, methods of the disclosure include topical administration, either to skin or to mucosal membranes such as those on the cervix and vagina. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The subject polypeptide therapeutic agents may be mixed under sterile conditions with a pharmaceutically acceptable carrier, and with any preservatives, buffers, or propellants which may be required. The ointments, pastes, creams and gels may contain, in addition to a subject conjugate of the disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a subject conjugate, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more conjugates in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more polypeptide therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydridcs). Depot injectable formulations are also prepared by entrapping the drug in liposomes or microemulsions which are compatible with body tissue.

In a preferred embodiment, the conjugates of the present disclosure are formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In another embodiment, the conjugates of the present disclosure are formulated for subcutaneous administration to human beings.

Note that, in certain embodiments, a particular formulation is suitable for use in the context of deliver via more than one route. Thus, for example, a formulation suitable for intravenous infusion may also be suitable for delivery via another route. However, in other embodiments, a formulation is suitable for use in the context of one route of delivery, but is not suitable for use in the context of a second route of delivery.

The amount of the conjugates of the disclosure which will be effective in the treatment of a tissue-related condition or disease can be determined by standard clinical techniques. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-5000 micrograms of the active conjugates per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

In certain embodiments, compositions of the disclosure, including pharmaceutical preparations, are non-pyrogenic. In other words, in certain embodiments, the compositions are substantially pyrogen free. In one embodiment the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in relatively large dosages and/or over an extended period of time (e.g., such as for the patient's entire life), even small amounts of harmful and dangerous endotoxin could be dangerous. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

The foregoing applies to any of the conjugates, compositions, and methods described herein. The disclosure specifically contemplates any combination of the features of such conjugates, compositions, and methods (alone or in combination) with the features described for the various pharmaceutical compositions and route of administration described in this section.

### VIII. Kits

In certain embodiments, the disclosure also provides a pharmaceutical package or kit comprising one or more containers filled with at least one conjugate of the disclosure. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both. In certain embodiments, the kit is a kit labeled for research purposes only.

In certain embodiments, the disclosure provides a kit comprises two or more containers in which one container comprises a polypeptide comprising an A44 or K39 peptide (e.g., an A44/K39 peptide portion) and a second container comprises a heterologous agent. The kit may optionally include reagents, optionally in one or more separate containers, suitable for making a conjugate comprising an A44 or K39 peptide portion (e.g., an A44/K39 peptide portion) and a heterologous agent portion. Such kits may optionally include instructions for making the conjugate, either instructions for chemical conjugation or instructions for making a fusion protein. The kit may include suitable linkers for interconnecting the two portions. Optionally, the kit may include directions for using the conjugate so made, such as in vitro for research purposes.

In certain embodiments, the kit includes additional materials to facilitate delivery of the subject conjugates. For example, the kit may include one or more of a catheter, tubing, infusion bag, syringe, and the like. In certain embodiments, the conjugate is packaged in a lyophilized form, and the kit includes at least two containers: a container comprising the lyophilized conjugate and a container comprising a suitable amount of water, buffer, or other liquid suitable for reconstituting the lyophilized material.

The foregoing applies to any of the conjugates, compositions, and methods described herein. The disclosure specifically contemplates any combination of the features of such conjugates, compositions, and methods (alone or in combination) with the features described for the various kits described in this section.

### EXEMPLIFICATION

The disclosure now being generally described, it will be more readily understood by reference to the following examples, which are included merely for purposes of illustration of certain aspects and embodiments of the present disclosure, and are not intended to limit the disclosure. For example, the particular constructs and experimental design disclosed herein represent exemplary tools and methods for validating proper function. As such, it will be readily apparent that any of the disclosed specific constructs and experimental plan can be substituted within the scope of the present disclosure.

### Example 1: Construction of A44/K39 Peptide Expression Plasmids and Expression of Recombinant Peptides

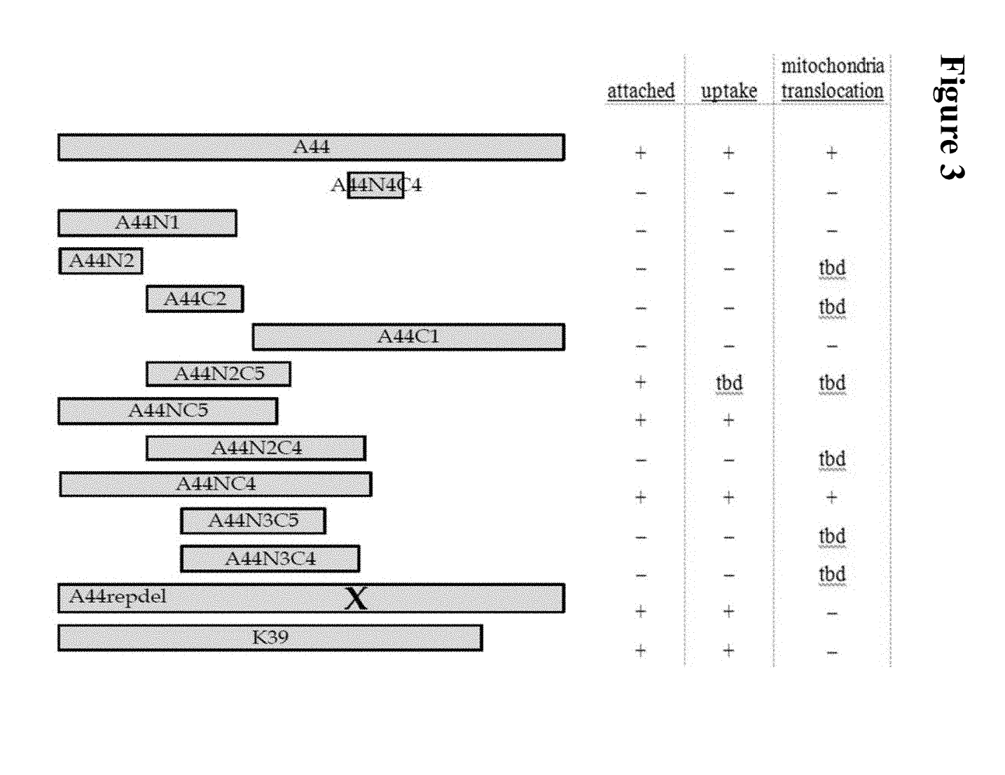

Construction and purification of the full-length A44 peptide and peptide fragments depicted in FIG. 3 were performed as previously described (Boisvert and Duncan (2008) supra). Briefly, various portions of the A44 peptide-encoding region were amplified from chromosomal DNA of P. gingivalis ATCC 33277 by PCR using various combinations of the forward and reverse primers shown in Table 1. PCR amplicons were ligated directly into pTOPO using the TOPO TA cloning kit (Invitrogen). Resulting pTOPO plasmids were digested with NdeI and XhoI; inserts were gel-purified using the Qiaquick kit (Qiagen) and then ligated with NdeI- and XhoI-digested pET22b vectors. E. coli BL21 (Novagen) were transformed with the ligated vectors and exposed to 1 mM isopropyl-a D-thiogalactopyranoside (IPTG) for 3-4 hours to induce expression of the recombinant peptide. Cells were lysed using 5 ml of lysis buffer per gram wet weight [100 mM NaH2PO4, 10 mM Tris, 6 M guanidine HCl (pH 8.0)]. Recombinant peptides were purified using Ni2+-NTA-agarose according to the supplier's instructions (Qiagen). We note that, for expression purposes, plasmids encoding an N-terminal methionine at the start of the A44 peptide fragments were made (e.g., the resulting A44 peptides and fragments also included an N-terminal methionine, such that the various A44 peptides and fragments of full length A44 peptides provided herein further included an N-terminal methionine at the start of each A44 peptide portion).

Similar methodologies can be utilized to generate expression constructs encoding K39-based peptides (full-length and fragments thereof) and produce recombinant K39-based peptides. Table 2 shows exemplary primers suitable for amplification of K39 regions from chromosomal DNA of P. gingivalis ATCC 33277 by PCR.

TABLE 1
Primers for construction
of A44-based peptides

| | |
|---|---|
| A44-N1 | TCATATGAGCGGTCAGGCCGAGATTG |
| A44-N2 | TCATATGGAAGGTGGTGGAAGCGAT |
| A44-N3 | TCATATGCCTTCTTGTTCCCCTAC |
| A44-N4 | TCATATGACCCCGAATCCAAATCC |
| A44-C1 | AGCTCGAGCTTGCCGTTGGCCTTGATCT 10 |
| A44-C2 | AGCTCGAGATCGCTTCCACCACCTTC |
| A44-C3 | AGCTCGAGTCTGCATTTTCCGGAACCG |
| A44-C4 | TCCTCGAGGGGATTCGGATTTGGATTC |
| A44-C5 | TCCTCGAGGGTACCATTAGGTGCATCCC |

TABLE 2
Primers for construction
of K39-based peptides

| | |
|---|---|
| K39-N1 | TACATATGGCCAACGAAGCCAAGGTTG |
| K39-C1 | AACTCGAGGCGCTTGCCATTGGCCT |
| K39-C4 | ATCTCGAGCGGATTCGGATTCGGATTCGGATTC |
| K39-C5 | ATCTCGAGGGATCCATTAGGTGCTACCCAC |

### Example 2: Identification of A44/K39 Peptide Domains Involved in Cellular Untake

Cellular uptake (i.e., cell penetration) of A44 peptides and fragments thereof was determined by Western blot of acid-washed cell lysates using methods set forth in Boisvert and Duncan (2008) supra. Briefly, 2 mg/ml recombinant A44-based peptide (full length or fragment) was incubated with HEp-2 epithelial cell monolayers for about 60 minutes. Cells were then washed three times with PBS followed by treatment with an acid wash solution (0.2 M acetic acid, 0.5 M NaCl, pH 2.5) to remove any externally attached peptide (5 min, RT). Cells were washed again (3×PBS) and lysed with water. Cellular lysates were harvested and the protein components separated by SDS-PAGE and transferred onto a nitrocellulose membrane. Presence of peptides in lysates was assayed with anti-His-tag antibodies, secondary HRP-antibodies and ECL according to the manufacturers' recommendation (Millipore). Results are summarized in FIG. 3. This is one example of methods that can be used to evaluate the cell uptake activity of various A44/K39 peptide portions, as well as conjugates of the disclosure.

Similar methodologies can be utilized to analyze cellular uptake of K39-based peptides and conjugates of the disclosure.

### Example 3: Identification of A44/K39 Peptide Domains Involved in Mitochondrial Targeting

Translocation to mitochondria was determined by fluorescent microscopy and cell fractionation in combination with Western Blots as shown previously (Boisvert and Duncan (2010) supra). Briefly, HEp-2 epithelial cells were incubated with recombinant an A44-derived peptide for about 60 minutes, washed and lysed with saponin buffer (1% (w/v) saponin in 10 mM Tris/HCl (pH 7.5). The supernatant in centrifuged samples contains cytoplasmic proteins. The resulting pellet was resuspended in saponin buffer+0.05% (v/v) Triton-X-100 and centrifuged again (max. speed, 5 min). The supernatant contained mitochondrial proteins as shown by Western Blot with anti-cytochrome C antibodies. Presence of A44-derived peptide in this fraction was visualized as described above. A44-derived peptide identified in this fraction can be further tested by confocal microscopy for co-localization with MitoTracker (Invitrogen) as described previously (Boisvert et al. (2010) supra).

Results for a variety of A44-derived peptides are summarized in FIG. 3. Detection of recombinant peptide within the cytoskeletal fraction indicated attachment. Peptides capable of attaching to the cell in this assay are indicated by a “+” in the column labeled “attached”. Detection of recombinant peptides within cytoplasmic and/or mitochondrial fractions indicated cellular uptake. Peptides capable of cellular uptake are indicated by a “+” in the column labeled “uptake”. Detection of recombinant peptides within mitochondrial fraction indicated mitochondrial translocation. Peptides capable of cellular uptake followed by translocation to the mitochondria are indicated by a “+” in the column labeled “mitochondria translocation”. Peptides for which one of the foregoing was not observed in this cell type and assay are indicated with “−”. This is one example of methods that can be used to evaluate the mitochondrial localization activity of various A44/K39 peptide portions, as well as conjugates of the disclosure.

Similar methodologies can be utilized to analyze mitochondrial targeting of K39-based peptides.

### Example 4: Identification of A44/K39 Peptide Domains that Induce Expression of Apoptosis Proteins Bcl-2 and Bcl-XL

Site-directed mutagenesis is employed to change amino acids within A44 or K39 derived peptides to either improve uptake and delivery or to interfere with upregulation factors involved in apoptosis. Interference with mitochondrial functions is measured using the Mitochondrial ToxGloTM Assay from Promega in combination with MTT assays and Western Blots to test for activation of caspases. RNAseq technology will be used to analyze if uptake and translocation of A44 or K39 peptides leads to gene expression changes. Using the TruSeq RNA Sample Preparation Kit v2 (Illumina), mRNA libraries are created from total RNA fractions of epithelial cells in which the A44 or K39 peptides have been internalized. Briefly, this involves purifying the poly-A containing mRNA molecules using oligo-dT attached magnetic beads. Following purification, the mRNA is fragmented into small pieces using divalent cations under elevated temperature. The cleaved RNA fragments are copied into first strand cDNA using reverse transcriptase and random primers. Second strand cDNA synthesis follows, using DNA Polymerase I and RNase H. The cDNA fragments then undergo an end repair process, the addition of a single ‘A’ base, and ligation to the adapters. The products are then purified and enriched with PCR to create the final cDNA library. DNA libraries are quantitated using qPCR, normalized to 10 nM and then pooled in equal volumes. These pooled libraries are analyzed using a NextSeq 500 to generate sequence information.

### Example 5: Uptake and Delivery Parameters of A44/K39 Peptide-Coupled Beads

“Red”-fluorescent latex beads (Sigma) are coated with A44 as described for latex beads before (Boisvert and Duncan (2010) supra). Real-time fluorescence microscopy is used to establish uptake and co-localization of A44/K39 peptide-coated beads with gfp-cytochrome C. Epithelial cells are transfected with a cytochrome C-gfp (Addgene) using standard procedures (Goldstein et al (2000) Nat Cell Biol 2(3): 156-162). Based on an established time-line for uptake of peptides, beads that co-localize with mitochondria are quantified used FACS analysis. MTT (3-(4,5-dimethylthiazol-2-yl)-2,5-diphenyltetrazolium bromide)-based assays showed that A44 is not toxic to human cells, and pre-incubation of cells with A44 protects them from staurosporine-induced apoptosis. The peptide is stable for days within epithelial cells without inducing host cell death.

### Example 6: Uptake and Delivery of Quantum-Dot-Labeled. PEGylated A44/K39 Peptides

PEGylation describes the process of attaching polyethylene glycol (PEG) to bioactive proteins of potential medical value and improves safety and efficacy of therapeutics. Suitable PEGylation reagents from Pierce are used to attach PEG of various lengths to an A44/K39-peptide. Qdot®655 ITKTM Amino (PEG) Quantum Dots (Molecular Probes) can also be used for conjugation of A44/K39 peptides, which allows us to easily follow uptake and trafficking of peptides of interest by confocal microscopy and live cell imaging. Fluorescent labeled PEG is used where possible (i.e. fragments containing cysteines). Fluorescent quantum dots are also used to label A44/K39-peptide fragments. Uptake and delivery are assessed as described above by real-time microscopy and FACS analysis.

### Example 7: Making Conjugated A44/K39 Peptides for Delivery of Various Cargos

### Covalent Conjugation of Small Molecules/Chemical Moieties

A44/K39 peptide portions described herein are covalently linked to various small inorganic or organic compounds having various therapeutic actions within a cell. For example, the highly biologically active antioxidant—α-tocopherol (vitamin E)—is conjugated to a recombinant A44/K39 peptide as recently described by Wang et al. (2007) Cancer Res. 67:3337-3344. Briefly, the peptide of interest is prepared according to the standard Fmoc protocol on a LIPS Vario Peptide synthesizer. All acylation reactions are carried out for 1 hour using a 10-fold excess of Fmoc-amino acids activated with TBTU (1 equivalent) in the presence of DIPEA (2 equivalents) and HOBt (1 equivalent). The NH2-terminal conjugation are carried out by activation of α-tocopherol with 1 equivalent of PyBOP in the presence of HOBt (1 equivalent) and DIPEA (2 equivalents). The conjugated peptide is cleaved from the resin using trifluoroacetic acid/triisopropylsilane/water (95:2.5:2.5) for 2 hours. The lipophilic peptide conjugate(s) is extracted with diethylether and validated by analysis with high-performance liquid chromatography (HPLC) and electrospray-mass spectrometry. A similar strategy is used to chemically conjugate the antioxidant L-ascorbic acid (vitamin C) to A44/K39 peptides as was previously described (Choi et al. (2009) BMB reports 42(11)743-746.).

### Example 8: Mitochondrial Translocation of A44/K39 Peptides in Cell Lines

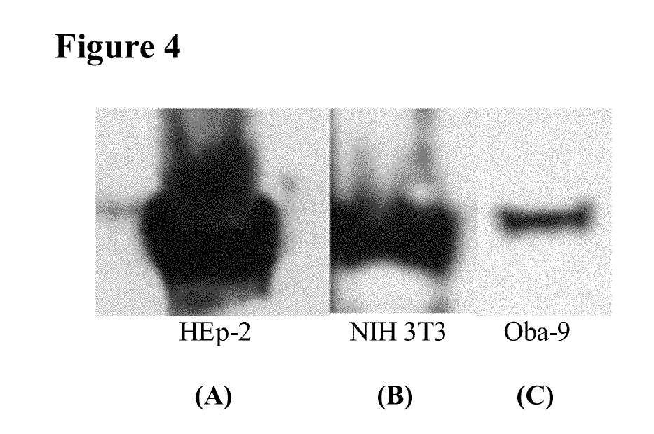

Translocation to mitochondria in cells of several cell lines (e.g., human epithelial type 2 (HEp-2), oral keratinocytes (Oba-9) and mouse fibroblasts (NIH 3T3) was determined by cell fractionation (Thermo Fisher mitochondria isolation kit, #89874) in combination with Western Blots. Briefly, cells were incubated with 2 mg/ml purified recombinant peptide for 60 min. Cell layers were washed, scraped and centrifuged at 700×g to pellet cells. Cells were resuspended in 400 μl buffer A and incubated for exactly 2 min on ice. 5 μl reagent B was added, and cells were vortexed and incubated for 5 min on ice. 400 μl reagent C was added, and the tubes were inverted several times and centrifuged for 10 min, 4° C. at 700×g. The supernatant was transferred to a fresh tube and centrifuged at 3000×g for 5 min (4° C.) to pellet mitochondria. The pellet was washed once and resuspended in SDS-loading dye. Samples were boiled and separated by SDS-PAGE. Gels were transferred onto nitrocellulose and blocked overnight with 5% milk ((w/v); in tris-buffered saline+0.05% Twen 20 (TBST). After 3 washes with TBST, the first antibody (anti-his-tag) was added (1:10000 in 5% milk) and the blot was incubated for ˜2 hours at room temperature. After another 3 washes with TBST, secondary horse-peroxidase-labeled secondary antibody was added and the blot was incubated for another 2 hours (in 5% milk in TBST) at room temperature. The blots were washed 3 times and subjected to enhanced chemiluminescence substrate (Millipore, according to the manufactures' instructions) and presence of recombinant peptides was detected on film. As shown in FIG. 4, the recombinant A44 peptide was capable of translocating to the mitochondria of human epithelial type 2 cells (HEp-2) (FIG. 4A), oral keratinocytes (Oba-9) (FIG. 4B), and mouse fibroblasts (NIH 3T3) (FIG. 4C). This is one example of methods that can be used to evaluate the mitochondrial localization activity of various A44/K39 peptide portions, as well as conjugates of the disclosure.

Similar methodologies can be utilized to analyze mitochondrial targeting of K39-based peptides.

### Example 9: Mitochondrial Translocation of A44/K39 Peptides in Gingival Tissue

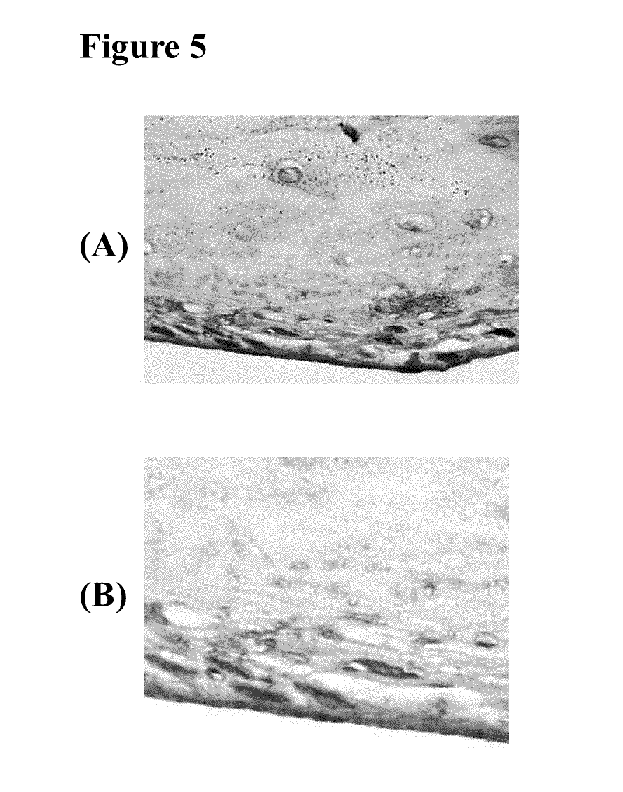

Translocation to mitochondria in a 3D gingival tissue model was determined. Briefly, three-dimensional (3D) gingival tissues were purchased from Mattek Corporation and inserts were immediately placed in a 6 well plate with 0.9 ml pre-warmed media (free of antibiotics, antifungals and hydrocortisone, provided by the manufacturer). 10 μg recombinant peptide A44 was added for 3 hours and inserts were washed, fixed with 4% neutral buffered formalin and embedded in paraffin. Antigen retrieval of paraffin sections was performed with 10 mM sodium citrate (pH 6.0) and endogenous peroxidase was blocked with 1% H₂O₂ in methanol. Sections were first blocked with horse serum for 2 hours are room temperature and then incubated with anti-TG2 (mouse) antibody overnight at 4° C. Secondary biotin-labeled anti-mouse antibody and substrate staining was performed with the Vectastain ABC kit and DAB from Vectorlabs according to their protocol. Sections were counterstained with hematoxylin and analyzed by light microscopy. The recombinant A44 peptide was capable of translocating to the mitochondria in a 3D gingival tissue as shown in FIG. 5A-B. This is one example of methods that can be used to evaluate the mitochondrial localization activity of various A44/K39 peptide portions, as well as conjugates of the disclosure.

Similar methodologies can be utilized to analyze mitochondrial targeting of K39-based peptides.

### Sequence Listing and Brief Description of Sequences:

In certain embodiments, the disclosure provides conjugates comprising an A44/K39 peptide portion comprising an amino acid sequence at least 80%, 85%, 90%, 95%, 96%, 97%, 98%, 99% or 100% identical to an A44 peptide, an A44 peptide fragment (e.g., a fragment of the A44 peptide), a K39 peptide, or a K39 peptide fragment (e.g., a fragment of the K39 peptide) disclosed herein, such as set forth in this sequence listing (e.g., set forth in SEQ ID NO: 1-26).

A44 peptide
(SEQ ID NO: 1)
SGQAEIVLEARDVWNDGSGYQILLDADHDQYGQVIPSDTHTLWPNCSVP
ANLFAPFEYTVPENADPSCSPTNMIMDGTASVNIPAGTYDFAIAAPQAN
AKIWIAGQGPTKEDDYVFEAGKKYHFLMKKMGSGDGTELTISEGGGSDY
TYTVYRDGTKIKEGLTATTFEEDGVAAGNHEYCVEVKYTAGVSPKVCKD
VTVEGSNEFAPVQNLTGSAVGQKVTLKWDAPNGTPNPNPNPNPNPNPGT
TTLSESFENGIPASWKTIDADGDGHGWKPGNAPGIAGYNSNGCVYSESF
GLGGIGVLTPDNYLITPALDLPNGGKLTFWVCAQDANYASEHYAVYASS
TGNDASNFTNALLEETITAKGVRSPEAIRGRIQGTWRQKTVDLPAGTKY
VAFRHFQSTDMFYIDLDEVEIKANGKLE
K39 peptide
(SEQ ID NO: 2)
ANEAKVVLAADNVWGDNTGYQFLLDADHNTFGSVIPATGPLFTGTASSN
LYSANFEYLIPANADPVVTTQNIIVTGQGEVVIPGGVYDYCITNPEPAS
GKMWIAGDGDNQPARYDDFTFEAGKKYTFTMRRAGMGDGTDMEVEDDSP
ASYTYTVYRDGTKIKEGLTATTFEEDGVAAGNHEYCVEVKYTAGVSPKV
CKDVTVEGSNEFAPVQNLTGSAVGQKVTLKWDAPNGTPNPNPNPNPGTT
TLSESFENGIPASWKTIDADGDGHGWKPGNAPGIAGYNSNGCVYSESFG
LGGIGVLTPDNYLITPALDLANGGKLTFWVCAQDANYASEHYAVYASST
GNDASNFTNALLEETITAKGVRSPEAIRGRIQGTWRQKTVDLPAGTKYV
AFRHFQSTDMFYIDLDEVEIKANGKRLE
A44NC4 (a fragment of A44 peptide)
(SEQ ID NO: 3)
SGQAEIVLEAHDVWNDGSGYQILLDADHDQYGQVIPSDTHTLWPNCSVP
ANLFAPFEYTVPENADPSCSPTNMIMDGTASVNIPAGTYDFAIAAPQAN
AKIWIAGQGPTKEDDYVFEAGKKYHFLMKFMGSGDGTELTISEGGGSDY
TYTVYRDGTKIKEGLTATTFEEDGVAAGNHEYCVEVKYTAGVSPKVCKD
VTVEGSNEFAPVQNLTGSAVGQKVTLKWDAPNGTPNPNPNPNPNPNP
K39NC4 (a fragment of K39 peptide)
(SEQ ID NO: 4)
ANEAKVVLAADNVWGDNTGYQFLLDADHNTFGSVIPATGPLFTGTASSN
LYSANFEYLIPANADPVVTTQNIIVTGQGEVVIPGGVYDYCITNPEPAS
GKMWIAGDGDNQPARYDDFTFEAGKKYTFTMRRAGMGDGTDMEVEDDSP
ASYTYTVYRDGTKIKEGLTATTFEEDGVAAGNHEYCVEVKYTAGVSPKV
CKDVTVEGSNEFAPVQNLTGSAVGQKVTLKWDAPNGTPNPNPNPNP
A44N2C4 (a fragment of A44 peptide)
(SEQ ID NO.: 5)
EGGGSDYTYTVYRDGTKIKEGITATTFEEDGVAAGNHEYCVEVKYTAGV
SPKVCKDVTVEGSNEFAPVQNLTGSAVGQKVTLKWDAPNGTPNPNPNPN
PNPNP
K39N2C4 (a fragment of K39 peptide)
(SEQ ID NO: 6)
EDDSPASYTYTVYRDGTKIKEGLTATTFEEDGVAAGNHEYCVEVKYTAG
VSPKVCKDVTVEGSNEFAPVQNLTGSAVGQKVTLKWDAPNGTPNPNPNP
NP
A44NC5 (a fragment of A44 peptide)
(SEQ ID NO: 7)
SGQAEIVLEAHDVWNDGSGYQILLDADHDQYGQVIPSDTHTLWPNCSVP
ANLFAPFEYTVPENADPSCSPTNMIMDGTASVNIPAGTYDFAIAAPQAN
AKIWIAGQGPTKEDDYVFEAGKKYHTLMKKMGSGDGTELTISEGGCSDY
TYTVYRDGTKIKEGLTATTFEEDGVAAGNHEYCVEVKYTAGVSPKVCKD
VTVEGSNEFAPVQNLTGSAVGQKVTLKWDAPNGT
K39NC5 (a fragment of K39 peptide)
(SEQ ID NO: 8)
ANEAKVVLAADNVVIGDNTGYULLDADHNTFGSVIPATGPLFTGTASSN
LYSANFEYLIPANADPVVTTQNIIVTGQGEVVIPGGVYDYCITNPEPAS
GKMWIAGDGDNQPARYDDFTFEAGKKYTFTMRRAGMGDGTDMEVEDDSP
ASYTYTVYRDGTKIKEGLTATTFEEDGVAAGNHEYCVEVKYTAGVSPKV
CKDVTVEGSNEFAPVQNLTGSAVGQKVTLKWDAPNGT
A44delrep (a fragment of A44 peptide)
(SEQ ID NO: 9)
SGQAEIVLEAHDVWNDGSGYQILLDADHDQYGQVIPSDTHTLWPNCSVP
ANLFAPFEYTVPENADPSCSPTNMIMDGTASVNIPAGTYDFAIAAPQAN
AKIWIAGQGPTKEDDYVFEAGKKYHFLMKKMGSGDGTELTISEGGGSDY
TYTVYRDGTKIKEGLTATTFEEDGVAAGNHEYCVEVKYTAGVSPKVCKD
VTVEGSNEFAPVQNLTGSAVGQKVTLKWDAPNGTGTTTLSESFENGIPA
SWKTIDADGDGHGWKPGNAPGIAGYNSNGCVYSESFGLGGIGVLTPDNY
LITPALDLPNGGKLTFWVCAQDANYASEHYAVYASSTGNDASNFTNALL
EETITAKGVRSPEAIRGRIQGTWRQKTVDLPAGTKYVAFRHFQSTDMFY
IDLDEVEIKANGKLE
K39delrep (a fragment of K39 peptide)
(SEQ ID NO: 10)
ANEAKVVLAADNVWGDNTGYQFLLDADHNTFGSVIPATGPLFTGTASSN
LYSANFEYLIPANADPVVTTQNIIVTGQGEVVIPGGVYDYCITNPEPAS
GKMWIAGDGDNQPARYDDFTFEAGKKYTFTMRRAGMGDGTDMEVEDDSP
ASYTYTVYRDGTKIKEGLTATTFEEDGVAAGNHEYCVEVKYTAGVSPKV
CKDVTVEGSNEFAPVQNLTGSAVGQKVTLKWDAPNGTGTTTLSESFENG
IPASWKTIDADGDGHGWKPGNAPGIAGYNSNGCVYSESFGLGGIGVLTP
DNYLITPALDLANGGKLTFWVCAQDANYASEHYAVYASSTGNDASNFTN
ALLEETITAKGVRSPEAIRGRIQGTWRQKTVDLPAGTKYVAFRHFQSTD
MFYIDLDEVEIKANGKR
A44N4C4
(SEQ ID NO: 11)
PNPNPNPNPNPNP
K39N4C4
(SEQ ID NO: 12)
PNPNPNPNP
A44C1 (a fragment of A44 peptide)
(SEQ ID NO: 13)
EGGGSDYTYTVYRDGTKIKEGLTATTFEEDGVAAGNHEYCVEVKYTAGV
SPKVCKDVTVEGSNEFAPVQNLTGSAVGQKVTLKWDAPNGTPNPNPNPN
PNPNPGTTTLSESFENGIPASWKTIDADGDGHGWKPGNAPGIAGYNSNG
CVYSESFGLGGIGVLTPDNYLITPALDLPNGGKLTFWVCAQDANYASEH
YAVYASSTGNDASNFTNALLFETTTAKGVRSPEATRGRIQGTWROKTVD
LPAGTKYVAFRHEQSTBMFYIDLDEVEIKANGKLE
K39C1 (a fragment of K39 peptide)
(SEQ ID NO: 14)
DDSPASYTYTVYRDGTKIKEGLTATTFEEDGVAAGNHEYCVEVKYTAGV
SPKVCKDVTVEGSNEFAPVQNLTGSAVGQKVTLKWDAPNGTPNPNPNPN
PGTTTLSESFENGIPASWKTIDADGDGHGWKPGNAPGIAGYNSNGCVYS
ESFGLGGIGVLTPDNYLITPALDLANGGKLTFWVCAQDANYASEHYAVY
ASSTGNDASNFTNALLEETITAKGVRSPEAIRGRIQGTWRQKTVDLPAG
TKYVAFRHFQSTDMFYIDLDEVETKANGKRLE
A44N3C4 (a fragment of A44 peptide)
(SEQ ID NO: 15)
PSCSPTNMIMDGTASVNIPAGTYDFAIAAPQANAKIWIAGQGPTKEDDY
VFEAGKKYHFLMKKMGSGDGTELTISEGGGSDYTYTVYRDGTKIKEGLT
ATTFEEDGVAAGNHEYCVEVKYTAGVSPKVCKDVTVEGSNEFAPVQNLT
GSAVGQKVTLKWDAPNGTPNPNPNPNPNPNP
K39N3C4 (a fragment of K39 peptide)
(SEQ ID NO: 16)
PVVTTQNIIVTGOGEVVIPGGVYDYCITNPEPASGKMWIAGDGDNQPAR
YDDFTFEAGKKYTFTMKRAGMGDGTDMEVEDDSPASYTYTVYRDGTKIK
EGLTATTFEEDGVAAGNHEYCVEVKYTAGVSPKVCKDVTVEGSNEFAPV
QNLTGSAVGQKVTLKWDAPNGTPNPNPNPNP
A44N3C5 (a fragment of A44 peptide)
(SEQ ID NO: 17)
PSCSPTNMIMDGTASVNIPAGTYDFAIAAPOANAKIWIAGQGPTKEDDY
VFEAGKKYHFLMKKMGSGDGTELTISEGGGSDYTYTVYRDGTKIKEGLT
ATTFEEDGVAAGNHEYCVEVKYTAGVSPKVCKDVTVEGSNEFAPVQNLT
GSAVGQKVTLKWDAPNGT
K39N3C5 (a fragment of K39 peptide)
(SEQ ID NO: 18)
PVVTTQNIIVTGQGEVVIPGGVYDYCITNPEPASGKMWIAGDGDNQPAR
YDDFTFEAGKKYTFTMRRAGMGDGTDMEVEDDSPASYTYTVYRDGTKIK
EGLTATTFEEDGVAAGNHEYCVEVKYTAGVSPKVCKDVTVEGSNEFAPV
QNLTGSAVGQKVTLKWDAPNGT
A44N1 (a fragment of A44 peptide)
(SEQ ID NO: 19)
SGQAETVLEARTWWNDGSGYQTLLDADHDQYGQVIPSDTHTLWPNCSVP
ANLFAPFEYTVPENADPSCSPTNMIMDGTASVNIPAGTYDFAIAAPQAN
AKIWIAGQGPTKEDDYVFEAGKKYHFLMKKMGSGDGTELTISEGGGSDL
E
K39N1 (a fragment of K39 peptide)
(SEQ ID NO: 20)
ANEAKVVLAADNVWGDNTGYQFLLDADHNTFGSVIPATGPLFTGTASSN
LYSANFEYLIPANADPVVTTQNIIVTGQGEVVIPGGVYDYCITNPEPAS
GKMWIAGDGDNQPARYDDFTFEAGKKYTFTMRRAGMGDGTDMEVEDDSP
AS
A44N2C5 (a fragment of A44 peptide)
(SEQ ID NO: 21)
EGGGSDYTYTVYRDGTKIKEGLTATTFEEDGVAAGNHEYCVEVKYTAGV
SPKVCKDVTVEGSNEFAPVQNLTGSAVGQKVTLKWDAPNGT
K39N2C5 (a fragment of K39 peptide)
(SEQ ID NO: 22)
DDSPASYTYTVYRDGTKIKEGLTATTFEEDGVAAGNHEYCVEVKYTAGV
SPKVCKDVTVEGSNEFAPVQNLTGSAVGQKVTLKWDAPNGTPNPNPNPN
P
A44C2 (a fragment of A44 peptide)
(SEQ ID NO: 23)
PSCSPTNMIMDGTASVNIPAGTYDFAIAAPQANAKIWIAGQGPTKEDDY
VFEAGKKYHFLMKKMGSGDGTELTISEGGGSDLE
K39C2 (a fragment of K39 peptide)
(SEQ ID NO: 24)
PVVTTQNIIVTGQGEVVIPGGVYDYCITNPEPASGKMWIAGDGDNQPAR
YDDFTFEAGKKYTFTMRRAGMGDGTDMEVEDDS
A44N2 (a fragment of A44 peptide)
(SEQ ID NO: 25)
SGQAEIVLEAHDVWNDGSGYQILLDADHDQYGQVIPSDTHTLWPNCSVP
ANLFAPFEYTVPENADLE
K39N2 (a fragment of K39 peptide)
(SEQ ID NO: 26)
ANEAKVVLAADNVWGDNTGYOFLLDADHNTFGSVIPATGPLFTGTASSN
LYSANFEYLIPANAD
RgpA
(SEQ ID NO: 27)
MNKFVSIALCSSLLGGMAFAQQTELGRNPNVRLLESTQQSVTKVQFRMD
NLKFTEVQTPKGMAQVPTYTEGVNLSEKGMPTLPILSRSLAVSDTREMK
VEVVSSKFIEKKNVLIAPSKGMIMRNEDPKKIPYVYGKSYSQNKFFPGE
IATLDDPFILRDVRGQVVNFAPLQYNPVTKTLRTYTETTVAVSETSEQG
KNILNKKGTFAGFEDTYKRMFMNYEPGRYTPVEEKQNGRMIVIVAKKYE
GDIKDFVDWKNQRGLRTEVKVAEDTASPVTANAIQQFVKQEYEKEGNDL
TYVLLVGDHKDIPAKITPGIKSDQVYGQIVGNDHYNEVFIGPFSCESKE
DLKTQTDRTTHYERNITTEDKWLGQALCIASAEGGPSADNGESDIQHEN
VIANLLTQYGYTKIIKCYDPGVTPKNIIDAFNGGISLVNYTGHGSETAW
GTSHFGTTHVKQLTNSNQLPFIFDVACVNGDFLFSMPCFAEALMRAQKD
GKPTGTVAIIASTINQSWASPMRGQDEMNEILCEKHPNNIKRTFGGVTM
NGMFAMVEKYKKDGEKMLDTWTVFGDPSLLVRTLVPTKMQVTAPAQINL
TDASVNVSCDYNGAIATISANGKMFGSAVVENGTATINLTGLTNESTLT
LTVVGYNKETVIKTINTNGEPNPYQPVSNLTATTQGQKVTLKWDAPSTK
TNATTNTARSVDGIRELVLLSVSDAPELLRSGQAEIVLEAHDVWNDGSG
YQILLDADHDQYGQVIPSDTHTLWPNCSVPANLFAPFEYTVPENADPSC
SPTNMIMDGTASVNIPAGTYDFAIAAPQANAKIWIAGQGPTKEDDYVFE
AGKKYHFLMKKMGSGDGTELTISEGGGSDYTYTVYRDGTKIKEGLTATT
FEEDGVATGNHEYQVEVKYTAGVSPKVCKDVTVEGSNEFAPVQNLTGSA
VGQKVTLKWDAPNGTPNPNPNPNPNPNPGTTTLSESFENGTPASWKTID
ADGDGHGWKPGNAPGIAGYNSNGCVYSESFGLGGIGVLTPDNYLITPAL
DLPNGGKLTFWVCAQDANYASEHYAVYASSTGNDASNFTNALLEETITA
KGVRSPEAIRGRIQGTWRQKTVDLPAGTKYVAFRHFQSTDMFYIDLDEV
EIKANGKRADFTETFESSTHGEAPAEWTTIDADGDGQGWLCLSSGQLDW
LTAHGGTNVVASFSWNGMALNPDNYLISKDVTGATKVKYYYAVNDGFPG
DHYAVMISKTGTNAGDFTVVFEETPNGINKGGARFGLSTEANGAKPQSY
WIERTVDLPAGTKYVAFRHYNCSDLNYILLDDIQFTMGGSPTPTDYTYT
VYRDGTKIKEGLTETTFEEDGVATGNHEYCVEVKYTAGVSPKECVNVTI
NPTQFNPVKNLKAQPDGGDVVLKWEAPSAKKTEGSREVKRIGDGLFVTI
EPANDVRANEAKVVLAADNVWGDNTGYQFLLDADHNTFGSVIPATGPLF
TGTASSNLYSANFEYLIPANADPVVTTQNIIVTGQGEVVIPGGVYDYCI
TNPEPASGKMWIAGDGGNQPARYDDFTFEAGKKYTFTMRRAGMGDGTDM
EVEDDSPASYTYTVYPDGTKIKEGLTETTYRDAGMSAQSHEYCVEVKYA
AGVSPKVCVDYIPDGVADVTAQKPYTLTVVGKTITVTQQGEAMIYDMNG
RRLAAGRNTVVYTAQGGYYAVMVVVDGKSYVEKLAVKLE
Kgp
(SEQ ID NO: 28)
MRKLLLLIAASLLGVGLYAQSAKIKLDAPTTRTTCTNNSFKQFDASFSF
NEVELTKVETKGGTFASVSIPGAFPTGEVGSPEVPAVRKLIAVPVGATP
VVRVKSFTEQVYSLNQYGSEKLMPHQPSMSKSDDPEKVPFVYNAAAYAR
KGFVGQELTQVEMLGTMRGVRIAALTINPVQYDVVANQLKVRNNIEIEV
SFQGADEVATQRLYDASFSPYFETAYKQLFNRDVYTDHGDLYNTPVRML
VVAGAKFKEALKPWLTWKAQKGFYLDVHYTDEAEVGTTNASTKAFIHKK
YNDGLAASAAPVFLALVGDTDVISGEKGKKTKKVTDLYYSAVDGDYFPE
MYTFRMSASSPEELTNIIDKVLMYEKATMPDKSYLEKALLIAGADSYWN
PKIGQQTIKYAVQYYYNQDHGYTDVYSYPKAPYTGCYSHLNTGVGFANY
TAHGSETSWADPSLTATQVKALTNKDKYFLAIGNCCVTAQFDYPQPCFG
EVMTRVKEKGAYAYIGSSPNSYWGEDYYWSVGANAVFGVQPTFEGTSMG
SYDATFLEDSYNTVNSIMWAGNLAATHAGNIGNITHIGAHYYWEAYHVL
GDGSVMPYRAMPKTNTYTLPASLPQNQASYSIQASAGSYVAISKDGVLY
GTGVANASGVATVNMTKQITENGNYDVVITRSNYLPVIKQIQAGEPSPY
QPVSNLTATTQGQKVTLKWDAPSAKKAEASREVKRIGDGLFVTIEPAND
VRANEAKVVLAADNVWGDNTGYQFLLDADHNTFGSVIPATGPLFTGTAS
SNLYSANFEYLIPANADPVVTTQNIIVTGQGEVVIPGGVYDYCITNPEP
ASGKMWIAGDGGNQPARYDDFTFEAGKKYTFTMRRAGMGDGTDMEVEDD
SPASYTYTVYRDGTKIQEGLTATTFEEDGVAAGNHEYCVEVKYTAGVSP
KVCKDVTVEGSNEFAPVQNLTGSAVGQKVTLKWDAPNGTPNPNPNPNPG
TTTLSESFENGIPASWKTIDADGDGHGWKPGNAPGIAGYNSNGCVYSES
FGLGGIGVLTPDNYLITPALDLPNGGKLTFWVCAQDANYASEHYAVYAS
STGNDASNFTNALLEETITAKGVRSPEAIRGRIQGTWRQKTVDLPAGTK
YVAFRHFQSTDMFYIDLDEVEIKANGKRADFTETFESSTHGEAPAEWTT
IDADGDGQDWLCLSSGQLDWLTAHGGTNVVASFSWNGMALNPDNYLISK
DVTGATKVKYYYAVNDGFPGDHYAVMISKTGTNAGDFTVVFEETPNGIN
KGGARFGLSTEANGAKPQSVWIERTVDLPAGTKYVAFRHYNCSDLNYIL
LDDIQFTMGGSPTPTDYTYTVYRDGTKIKEGLTETTFEEDGVATGNHEY
CVEVKYTAGVSPKVCVNVTINPTQFNPVKNLKAQPDGGDVVLKWEAPSG
KRGELLNEDFEGDAIPTGWTALDADGDGNNWDITLNEFTRGERHVLSPL
RASNVAISYSSLLQGQEYLPLTPNNFLITPKVEGAKKITYKVGSPGLPQ
WSHDHYALCISKSGTAAADFEVIFEETMTYTQGGANLTREKDLPAGTKY
VAFRHYNCTDVLGIMIDDVVITGEGEGPSYTYTVYRDGTKIQEGLTETT
YRDAGMSAQSHEYCVEVKYAAGVSPKVCVDYIPDGVADVTAQKPYTLTV
VGKTITVTCQGEAMIYDMNGRRLAAGRNTVVYTAQGGYYAVMVVVDGKS
YVEKLAIK

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A conjugate comprising
i) an A44/K39 peptide portion capable of entering a cell by endocytosis and translocating to mitochondria, and
ii) a heterologous agent covalently associated therewith, wherein the heterologous agent is not a fluorescent protein or a fluorescent dye; and
wherein the conjugate does not comprise a catalytic domain of a gingipain protein.

2. The conjugate of claim 1, wherein the conjugate does not comprise a gingipain adhesin peptide other than an A44/K39 peptide.

3. The conjugate of any proceeding claim, wherein (i) comprises a fragment of an A44 or K39 peptide capable of entering a cell by endocytosis and translocating to mitochondria.

4. The conjugate of any proceeding claim, wherein the fragment of an A44 or K39 peptide comprises at least 50 amino acid residues.

5. The conjugate of any proceeding claim, wherein the conjugate does not include a full length, native A44 or K39 peptide.

6. The conjugate of any proceeding claim, wherein neither (i) nor the conjugate comprise the amino acid sequence set forth in SEQ ID NO.: 1 and/or SEQ ID NO.: 2.

7. The conjugate of any proceeding claim, wherein, if the conjugate comprises an epitope tag it further comprises an additional heterologous agent.

8. The conjugate of any proceeding claim, wherein the conjugate further comprises a moiety to increase in vivo stability or in vivo half life, or wherein the heterologous agent comprises a moiety to increase in vivo stability or in vivo half life.

9. The conjugate of claim 8, wherein the moiety comprises a nonproteinaceous polymer, such as polyethylene glycol, polypropylene glycol, or polyoxyalkylenes.

10. The conjugate of any proceeding claim, wherein the heterologous agent is N-terminal to the peptide.

11. The conjugate of any proceeding claim, wherein the heterologous agent is C-terminal to the peptide.

12. The conjugate of any proceeding claim, wherein the conjugate further comprises an Fc portion of an antibody.

13. The conjugate of any proceeding claim, further comprising a linker interconnecting the A44/K39 peptide portion and the heterologous agent.

14. The conjugate of any proceeding claim, wherein (i) and (ii) are interconnected by a linker.

15. The conjugate of any proceeding claim, wherein the A44/K39 peptide portion and heterologous agent are directly interconnected without a linker.

16. The conjugate of any preceding claim, wherein the A44/K39 peptide portion comprises an amino acid sequence at least 80% identical to SEQ ID NO.: 1, or a fragment thereof capable of entering a cell by endocytosis and translocating to mitochondria.

17. The conjugate of any preceding claim, wherein the A44/K39 peptide portion comprises an amino acid sequence at least 80% identical to SEQ ID NO.: 2, or a fragment thereof capable of entering a cell by endocytosis and translocating to mitochondria.

18. The conjugate of any preceding claim, wherein the A44/K39 peptide portion comprises an amino acid sequence at least 80% identical to SEQ ID NO.: 3 or SEQ ID NO.: 4, or a fragment thereof capable of entering a cell by endocytosis and translocating to mitochondria.

19. The conjugate of any preceding claim, wherein the A44/K39 peptide portion comprises an amino acid sequence at least 80% identical to SEQ ID NO.: 5 or SEQ ID NO.: 6, or a fragment thereof capable of entering a cell by endocytosis and translocating to mitochondria.

20. The conjugate of any preceding claim, wherein the cell is an epithelial cell.

21. The conjugate of any proceeding claim, wherein the heterologous agent is a protein, peptide, polynucleotide, oligonucleotide or small organic molecule.

22. The conjugate of any preceding claim, wherein the conjugate comprises a fusion protein comprising the A44/K39 peptide portion and the heterologous agent.

23. The conjugate of claim 22, wherein the fusion protein is produced using a recombinant vector encoding both the A44/K39 peptide portion and the heterologous agent.

24. The conjugate of claim 23, wherein the recombinant vector further comprises a peptide linker between the A44/K39 peptide portion and the heterologous agent.

25. The conjugate of any of claims 1-21, wherein the A44/K39 peptide portion and the heterologous agent are chemically conjugated.

26. The conjugate of any of claims 1-21, wherein (i) and (ii) are chemically conjugated.

27. The conjugate of claim 25 or 26, wherein the heterologous agent is N-terminal to the peptide.

28. The conjugate of claim 25 or 26, wherein (ii) is C-terminal to (i).

29. The conjugate of any preceding claim, wherein the heterologous agent is a pharmaceutical agent.

30. The conjugate of any preceding claim, wherein the heterologous agent is a nutraceutical agent.

31. The conjugate of claim 30, wherein the nutraceutical agent is vitamin E or vitamin C.

32. The conjugate of any preceding claim, wherein the heterologous agent is a therapeutic agent or a therapeutically active agent, such as an antioxidant.

33. The conjugate of any preceding claim, wherein the heterologous agent is endogenously present or active in mitochondria.

34. The conjugate of any one of claims 1-30, 32 and 33, wherein the heterologous agent is a polypeptide or peptide, and wherein the polypeptide or peptide is selected from: an enzyme, a cofactor, a cytochrome, an antibody or a growth factor.

35. The conjugate of any of claims 1-30, 32, 33 and 34, wherein the heterologous agent is a polynucleotide or oligonucleotide.

36. The conjugate of any preceding claim, wherein the heterologous agent is suitable for treating a mitochondrial disease or condition.

37. A composition comprising the conjugate of any preceding claim and one or more pharmaceutically acceptable carriers and/or excipients.

38. A nucleic acid construct, comprising a nucleotide sequence that encodes (i) the conjugate of any preceding claim or (ii) the peptide portion of the conjugate of any preceding claim, wherein the nucleic acid construct encodes a conjugate capable of entering a cell by endocytosis and localizing to mitochondria.

39. A host cell comprising the nucleic acid of claim 38.

40. A method of producing the conjugate of claim 38, comprising culturing the host cell of claim 39 under conditions for producing said conjugate.

41. The method of claim 40, further comprising isolating said conjugate.

42. A method for treating a mitochondrial disorder or mitochondrial dysfunction in a cell, the method comprising administering an effective amount of the conjugate according to any one of claims 1-37.

43. A method for delivering a heterologous agent to mitochondria in a cell, the method comprising administering an effective amount of the conjugate of any one of claims 1-37.

44. A method of preventing or decreasing cell death in a cell, the method comprising administering an effective amount of the conjugate of any one of claims 1-37.

45. A method of preventing or decreasing oxidative stress in a cell, the method comprising administering an effective amount of the conjugate of any one of claims 1-37.

46. A method of reducing the number of mitochondria undergoing mitochondrial permeability transitioning (MPT) or decreasing mitochondrial permeability transitioning in a cell, the method comprising administering an effective amount of the conjugate of any one of claims 1-37.

47. The method according to any one of claims 42-46, wherein administering the conjugate results in increased expression of one or more anti-apoptotic genes, e.g., bcl-2, bcl-XL, NFkB, and mcl-1, relative to an untreated cell.

48. The method according to any one of claims 42-47, wherein administering the conjugate results in decreased expression of one or more pro-apoptotic genes, e.g., bax, bak and TNFa, relative to an untreated cell.

49. The method according to any one of claims 42-48, wherein administering the conjugate results in increased expression of one or more pro-survival genes, e.g., rhoA, stat3, and cIAP1, relative to an untreated cell.

50. The method according to any one of claims 42-49, wherein the cell is an epithelial cell.

51. The method according to any one of claims 42-50, wherein the cell is a human cell.

52. The method of any of claims 42-51, wherein the cell is characterized by a mitochondrial dysfunction and/or is a cell from or of a subject having a mitochondrial disorder or condition.

53. The method of any of claims 42-52, wherein the cell is in a subject.

54. The method according to any one of claims 42-53, wherein the subject is a subject in need of treatment for a mitochondrial disease or condition.

55. The method according to claim 54, wherein the disease or condition is characterized by increased oxidative damage in a cell.

56. The method according to claim 54 or 55, wherein the disease or condition is characterized by increased cell death.

57. The method according to any one of claims 54-56, wherein the disease or condition is characterized by degeneration e.g., cardiovascular diseases such as atherosclerosis, ischemia/reperfusion injury, heart failure, stroke, and traumatic brain injury; neurodegenerative diseases such as Alzheimer's disease, Parkinson's disease, Huntington's disease, amyotrophic lateral sclerosis (ALS), autism, and muscular dystrophy; chronic autoimmune inflammatory diseases such as rheumatoid arthritis; and metabolic diseases such as diabetes and obesity.

58. The method according to claim 57, wherein the heterologous agent of the conjugate promotes cell survival.

59. The method according to any one of claim 54, wherein the disease or condition is characterized by a hyperproliferative state, e.g., cancer.

60. The method according to claim 59, wherein the heterologous agent of the conjugate promotes cell death.

61. The method according to any one of claims 42-60, wherein the conjugate is administered orally, topically, intranasally, systemically, intravenously, subcutaneously, intramuscularly, or transdermally.

62. The method according to any one of claims 42-61, wherein the conjugate is combined with a pharmaceutically acceptable carrier prior to administration to the subject.

63.-86. (canceled)
